(19) United States
(12) Patent Application Publication
(10) Pub. No.: US 2026/0225130 A1
(43) Pub. Date: Aug. 6, 2026

(54) COMPOUND, COMPOSITION, SURFACE TREATMENT AGENT, PELLET, ARTICLE, AND METHOD FOR MANUFACTURING ARTICLE

(71) Applicant: AGC Inc., Tokyo (JP)

(72) Inventors: Koki WATANABE, Tokyo (JP); Eiichiro ANRAKU, Tokyo (JP); Masayuki HARAGUCHI, Tokyo (JP)

(73) Assignee: AGC Inc., Tokyo (JP)

(21) Appl. No.: 19/565,780

(22) Filed: Mar. 13, 2026

Related U.S. Application Data
(63) Continuation of application No. PCT/JP2024/032852, filed on Sep. 13, 2024.

(30) Foreign Application Priority Data
Sep. 15, 2023 (JP) 2023-150604
Feb. 9, 2024 (JP) 2024-018476
Apr. 3, 2024 (JP) 2024-060422
Jun. 21, 2024 (WO) PCT/JP2024/022675

(51) Int. Cl. B05D 1/00; B05D 5/08

(52) U.S. Cl. CPC B05D 1/60; B05D 5/08

(57) ABSTRACT

A compound capable of forming a surface-treated layer with excellent acid resistance, a composition and a surface treatment agent comprising such a compound, an article including a surface-treated layer with excellent acid resistance, and a method for manufacturing such an article are provided. A compound represented by following Formula (1)

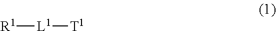

in Formula (1), R¹ is [(CH₃)₃Q¹-R²-]ₘ₁Q²(CH₃)₃₋ₘ₁—; Q¹ and Q² are each independently C or Si; R² is an alkylene group having 1 to 6 carbon atoms; m1 is an integer from 0 to 3; L¹ is an alkylene group having 13 or more carbon atoms; T¹ is —Si(R¹¹)ₘ₂L₃₋ₘ₂; R¹¹ is a hydrocarbon group; L is a hydrolyzable group, a group having a hydrolyzable group, or a hydroxyl group; and m2 is an integer from 0 to 2.

### INCORPORATION BY REFERENCE

This application is based upon and claims the benefit of priority from Japanese Patent Application No. 2023-150604 filed on Sep. 15, 2023, Japanese Patent Application No. 2024-018476 filed on Feb. 9, 2024, Japanese Patent Application 2024-060422 filed on Apr. 3, 2024, International Patent Application No. PCT/JP2024/022675 filed on Jun. 21, 2024, and International Patent Application No. PCT/JP2024/032852 filed on Sep. 13, 2024, the disclosure of which is incorporated herein in its entirety by reference.

### BACKGROUND

The present disclosure relates to a compound, a composition, a surface treatment agent, a pellet, an article, and a method for manufacturing an article.

In recent years, there has been a demand for a technique prevent fingerprint adhesion to the surface of an article and to facilitate the removal of contaminants (e.g., smudges) in order to improve properties of the article such as its appearance and visibility. As a specific method, a method for treating the surface of an article by using a surface treatment agent is known.

Patent Literature 1 discloses a specific siloxane group-comprising silane compound having a divalent linear organopolysiloxane group and a hydrolyzable silyl group as a surface treatment agent.
[Patent Literature 1] International Patent Publication No. 2023/017830

### SUMMARY

It has been desired to further improve the acid resistance of a surface-treated layer in view of the durability thereof.

An object of the present disclosure is to provide a compound capable of forming a surface-treated layer with excellent acid resistance, a composition and a surface treatment agent comprising such a compound, an article including a surface-treated layer with excellent acid resistance, and a method for manufacturing such an article.

The present disclosure includes the following aspects.

[1]

A compound represented by following Formula (1)

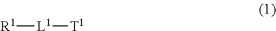

in Formula (1),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3,
L¹ is an alkylene group having 13 or more carbon atoms, and may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms,
T¹ is —Si(R¹¹)ₘ₂L₃₋ₘ₂,
R¹¹ is each independently a hydrocarbon group,
L is each independently a hydrolyzable group, a group having a hydrolyzable group, or a hydroxyl group, and
m2 is an integer from 0 to 2.
   [2]

The compound according to Item [1], wherein the number of carbon atoms of L¹ is 36 or less.

[3]

The compound according to Item [1] or [2], wherein L¹ is an alkylene group having 13 to 36 carbon atoms.

[4]

The compound according to any one of Items [1] to [3], wherein R¹ is Q²(CH₃)₃—.

[5]

The compound according to any one of Items [1] to [3], wherein L is represented by following Formula (G1).

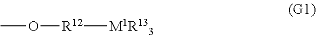

where
R¹² is an alkylene group, (Si(CH₃)₂O)ₘ₃, or a combination of an alkylene group and (Si(CH₃)₂O)ₘ₃,
M¹ is a carbon atom or a silicon atom,
R¹³ is each independently a hydrogen atom or an alkyl group which may have a branch, and each of at least two of R¹³ is an alkyl group which may have a branch, and
m3 is an integer from 1 to 12.
   [6]

The compound according to Item [5], wherein R¹² is a linear alkylene group.

[7]

The compound according to Item [5] or [6], wherein M¹R¹³₃ is CH(CH₃)₂, C(CH₃)₃, or Si(CH₃)₃.

[8]

The compound according to any one of Items [1] to [7], wherein the compound is in a solid state at 25° C.

[9]

The compound according to any one of Items [1] to [7], wherein the compound is in a liquid state at 25° C.

[10]

A composition comprising a compound according to any one of Items [1] to [9] and a liquid medium.

[11]

A surface treatment agent comprising a compound according to any one of Items [1] to [9].

[12]

The surface treatment agent according to Item [11], further comprising a liquid medium.

[13]

The surface treatment agent according to Item [11] or [12], wherein the surface treatment agent is one for vacuum deposition.

[14]

The surface treatment agent according to Item [12], wherein the surface treatment agent is one for wet coating.

[15]

A pellet comprising the surface treatment agent according to Item [11] or [12].

[16]

A method for manufacturing an article including a surface-treated layer formed on a substrate by performing a surface treatment on the substrate by using a surface treatment agent according to any one of Items [11] to [14].

[17]

An article comprising a substrate, and a surface-treated layer disposed on the substrate, a surface of the surface-treated layer being treated with a surface treatment agent according to any one of Items [11] to [14].

[18]

The article according to Item [17], wherein the article is an optical member.

[19]

The article according to Item [17] or [18], wherein the article is a display or a touch panel.

[20]

A compound represented by following Formula (13)

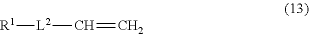

in Formula (13),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3, and
L² is an alkylene group having 11 or more carbon atoms, and may have one or more bonds selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.

According to the present disclosure, it is possible to provide a compound capable of forming a surface-treated layer with excellent acid resistance, a composition and a surface treatment agent comprising such a compound, an article including a surface-treated layer with excellent acid resistance, and a method for manufacturing such an article.

The above and other objects, features and advantages of the present disclosure will become more fully understood from the detailed description given hereinbelow.

### DESCRIPTION OF EMBODIMENTS

A numerical value range specified by using “-” in the specification of the present disclosure includes numerical values before and after “-” as a lower limit value and an upper limit value, respectively, of the range.

In numerical ranges described in a stepwise manner in the present specification, the upper or lower limit value of one numerical range may be replaced with the upper or lower limit value of another numerical range described in a stepwise manner. Further, in numerical ranges described in a stepwise manner in the present specification, the upper or lower limit value of a numerical range may be replaced with values shown in Examples.

In the specification of the present disclosure, the “surface-treated layer” refers to a layer that is formed on the surface of a substrate by a surface treatment.

In the specification of the present disclosure, “Me” may refer to a methyl group; “tBu” may refer to a tert-butyl group; and “TMS” may refer to a trimethylsilyl group.

In the specification of the present disclosure, when a compound or a group is represented by a specific formula (X), the compound or the group represented by this formula (X) may be expressed as a compound (X) or a compound X, and a group (X) or a group X, respectively.

Note that when the same reference numerals are present in one chemical formula, the same reference numerals may represent the same structure or may represent different structures within a specified range.

The bonding direction of a divalent group shown in the specification of the present disclosure is not limited to any particular directions unless otherwise specified. For example, when Y is —COO— in a compound represented by a formula “X—Y—Z”, Y may be —CO—O— or —O—CO—. Further, the aforementioned compound may be “X—CO—O—Z” or “X—O—CO—Z”.

### [Compound]

A compound according to the present disclosure is a compound represented by Formula (1) (which will be described later). When the compound (1) is used as a surface treatment agent, a surface-treated layer with excellent acid resistance can be formed.

Although the reason for this fact is not clear, it is presumed as follows. Roughly speaking, a compound according to the present disclosure has a structure of “group comprising TMS or tBu-specific long-chain alkylene group-reactive silyl group”. Since the compound according to the present disclosure has a reactive silyl group having a high adhesive property to a substrate, a surface-treated layer that tightly adheres to the surface of the substrate can be formed. Further, it is presumed that since the compound according to the present disclosure contains a long-chain alkylene having a reactive silyl group at an end, the molecules of the compound are densely arranged on the substrate.

Further, since TMS or tBu is disposed on the air interface side of the surface-treated layer of the compound according to the present disclosure, the compound has excellent water repellency and excellent oil repellency, and its fingerprint wiping-out property is improved. Because of these features, if an acid adheres to the surface-treated layer, the acid is prevented from penetrating into the substrate side, so that the durability, such as acid resistance, water resistance, and hot-water resistance, of the surface-treated layer is improved. Further, since the molecules of the compound disclosed herein are densely arranged on the substrate, the abrasion resistance is also further improved.

A compound according to the present disclosure will be described hereinafter in detail.

### <Compound (1)>

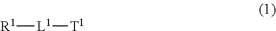

In Formula (1),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3,
L¹ is an alkylene group having 13 or more carbon atoms, and may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms,
T¹ is —Si(R¹¹)ₘ₂L₃₋ₘ₂,
R¹¹ is each independently a hydrocarbon group,
L is each independently a hydrolyzable group, a group having a hydrolyzable group, or a hydroxyl group, and
m2 is an integer from 0 to 2.

In Formula (1), R¹ is [(CH₃)₃Q¹-R²-]ₘ₁Q²(CH₃)₃₋ₘ₁—. Q² is bonded to L¹.

Q¹ and Q² are each independently C or Si.

R² is an alkylene group having 1 to 6 carbon atoms. Since the compound contains R², the steric hindrance is alleviated even when two or more (CH₃)₃Q¹ are present at the end of the compound (1). As a result, the rotation of a plurality of (CH₃)₃Q¹ is ensured, so that a surface-treated layer with excellent water repellency and excellent oil repellency can be formed.

The alkylene group in R² may be a straight chain or may have a branch. Specific examples of R² include —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH(CH₃)—, —CH(CH₃)CH₂—, —CH(CH₃)CH₂CH₂—, —CH(CH₂CH₃)CH₂CH₂—, —CH₂C(CH₂)₂CH₂—, —CH₂CH(CH₃)CH₂CH₂CH₂—, and —CH₂CH₂CH(CH₃)CH₂CH₂—. When R² is an alkylene group having a branch, the number of carbon atoms of the branched chain is preferably 1 or 2 and more preferably 1 (i.e., a methyl group) in order to further improve the abrasion resistance. The number of carbon atoms of the alkylene group in R² is preferably 1 to 5 and more preferably 1 to 4.

m1 is an integer from 0 to 3. In order to further improve the abrasion resistance, the acid resistance, and the hot-water resistance, m1 is preferably 0 to 2 and more preferably 0 or 1. When m1 is 0, R¹ is represented by the following Formula (2), and is specifically TMS or tBu.

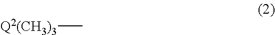

Examples of R¹ include groups shown below. Note that * represents a position of a bond with L¹.

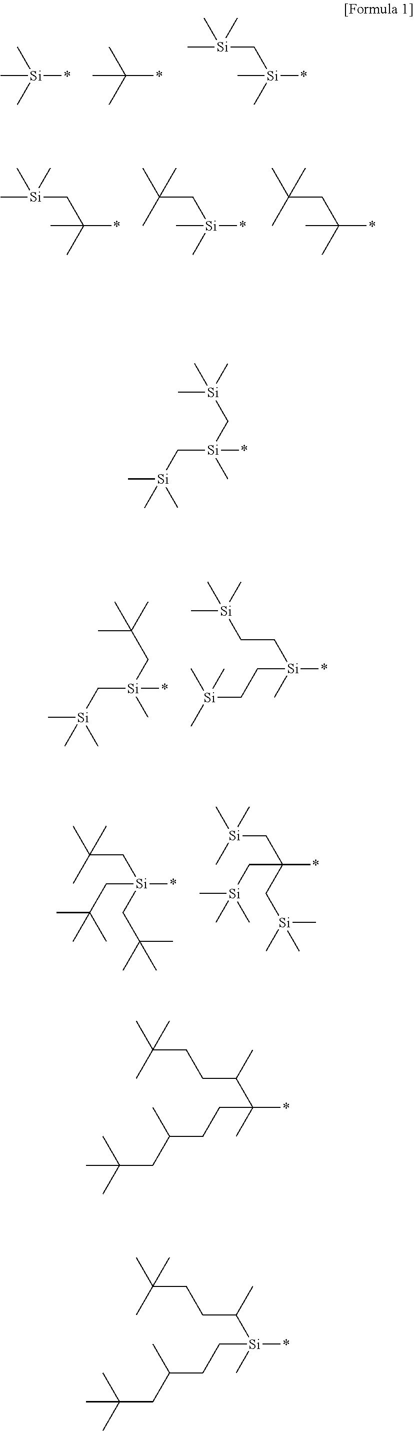

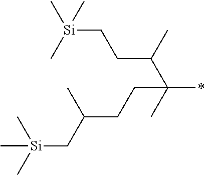

In Formula (1), L¹ is an alkylene group having 13 or more carbon atoms, and may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.

Since L¹ is an alkylene group having 13 or more carbon atoms, it is possible to form a surface-treated layer having not only excellent acid resistance but also excellent hot-water resistance and excellent friction resistance. It is sufficient if the number of carbon atoms of L¹ is 13 or larger, and in order to further improve the acid resistance, the hot-water resistance, and the friction resistance of the surface-treated layer, it is preferably 15 or larger and more preferably 19 or larger. Meanwhile, the upper limit of the number of carbon atoms of L¹ is not limited to any particular numbers. For example, it is sufficient if it is 100 or less, and in view of the ease of the manufacturing of the compound (1), it is preferably 36 or less. Note that carbons (e.g., the carbon in —C(═O)O—) of each of the above-described groups are not included in the aforementioned number of carbon atoms of the alkylene group.

The alkylene group of L¹ may be a straight chain or may have a branch. In the case of an alkylene group having a branch, the number of carbon atoms of the branched chain is preferably 1 or 2 and more preferably 1 (i.e., a methyl group). On the other hand, in order to further improve the acid resistance, the hot-water resistance, and the friction resistance of the surface-treated layer, L¹ is preferably a straight-chain alkylene group.

The alkylene group of L¹ may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.

In order to further improve the acid resistance, the hot-water resistance, and the friction resistance of the surface-treated layer, the number of the aforementioned groups in L¹ is preferably 0 to 2 and more preferably 0 or 1.

When L¹ has the above-described group, this group is preferably located on the T¹ side of the center of L¹ in order to further improve the acid resistance, the hot-water resistance, and the friction resistance of the surface-treated layer.

As L¹, a group represented by the following Formula (3) is preferred.

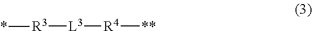

In Formula (3),
R³ is a linear alkylene group having 6 or more carbon atoms,
R⁴ is a single bond or a linear alkylene group having 1 to 6 carbon atoms,
L³ is a single bond, —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, or a phenylene group,
when L³ is a single bond, R⁴ is a single bond, and R³ is a linear alkylene group having 13 or more carbon atoms,
when L³ is not a single bond, R⁴ is a linear alkylene group having 1 to 6 carbon atoms, and the total number of carbon atoms of R³ and R⁴ is 13 or larger, and
* represents a position of a bond with R¹ with, and ** represents a position of a bond with T¹.

When L³ is a single bond, the number of carbon atoms of R³ is preferably 13 or larger, more preferably 15 or more, and still more preferably 17 or larger in order to further improve the acid resistance, the hot-water resistance, and the friction resistance of the surface-treated layer. Further, in view of the ease of the manufacturing, the number of carbon atoms of R³ is preferably 36 or less.

When L³ is any of various bonds other than the single bond, the number of carbon atoms of R³ is preferably 12 or larger, more preferably 14 or larger, and still more preferably 16 or larger in order to further improve the acid resistance, the hot-water resistance, and the friction resistance of the surface-treated layer. Further, in view of the ease of the manufacturing of the compound (1), the number of carbon atoms of R⁴ is preferably 2 to 6 and more preferably 3 to 6.

In order to further improve the acid resistance, the hot-water resistance, and the friction resistance of the surface-treated layer, L³ is preferably a single bond.

Examples of L¹ include groups shown below. Note that * represents a position of a bond with R¹, and ** represents a position of a bond with T¹. n1 is each independently an integer from 5 or larger, and is preferably 32 or less.

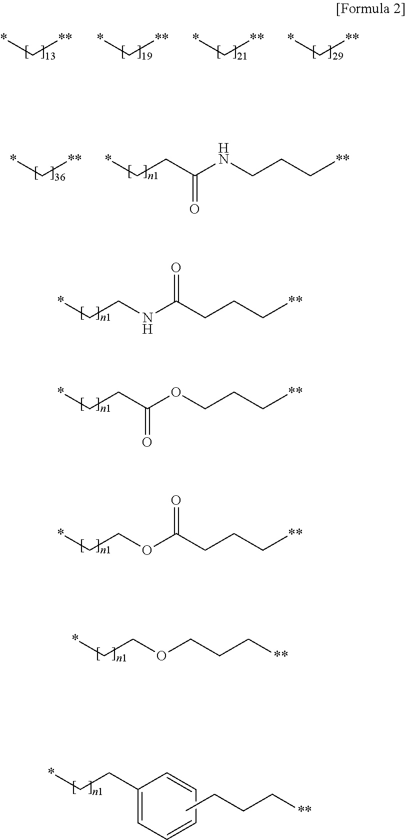

In Formula (1), T¹ is —Si(R¹¹)ₘ₂L₃₋ₘ₂,
R¹¹ is each independently a hydrocarbon group,
L is each independently a hydrolyzable group, a group having a hydrolyzable group, or a hydroxyl group, and
m2 is an integer from 0 to 2.

R¹¹ is each independently a hydrocarbon group.

Examples of the hydrocarbon group of R¹¹ include an aliphatic hydrocarbon group and an aromatic hydrocarbon group. In particular, the hydrocarbon group is preferably an aliphatic hydrocarbon group and more preferably an alkyl group. The alkyl group may be any of a linear alkyl group, a branched alkyl group, or a cyclic alkyl group, and is preferably a linear alkyl group. The number of carbon atoms of the alkyl group is preferably 1 to 10, more preferably 1 to 6, and still more preferably 1 to 4. The hydrocarbon group represented by R¹¹ is more preferably a methyl group, an ethyl group, an n-propyl group, or an n-butyl group, and still more preferably a methyl group.

L is each independently a hydrolyzable group, a group having a hydrolyzable group, or a hydroxyl group.

The hydrolyzable group is a group which becomes a hydroxyl group through a hydrolysis reaction. That is, a hydrolyzable silyl group represented by Si-L becomes a silanol group represented by Si—OH through a hydrolysis reaction. Such silanol groups further react with each other and form a Si—O—Si bond. Further, such a silanol group has a dehydration condensation reaction with a silanol group derived from an oxide present on the surface of the substrate, and thereby can form a Si—O—Si bond.

Examples of hydrolyzable groups include an alkoxy group, an aryloxy group, a halogen atom, an acyl group, an acyloxy group, a dialkylamino group, and an isocyanato group (—NCO). The alkoxy group is preferably an alkoxy group having 1 to 4 carbon atoms because the reactivity becomes excellent. The aryloxy group is preferably an aryloxy group having 3 to 10 carbon atoms. Note that the aryl group of the aryloxy group includes a heteroaryl group. The halogen atom is preferably a chlorine atom. The acyl group is preferably an acyl group having 1 to 6 carbon atoms. The acyloxy group is preferably an acyloxy group having 1 to 6 carbon atoms.

In order to improve the storage stability of a liquid composition comprising the compound (1), L is preferably an alkoxy group having 5 or more carbon atoms. In particular, L is preferably an alkoxy group having a tertiary carbon atom, a quaternary carbon atom, a trialkylsilyl group, or a polydimethylsiloxane group, and more preferably a group having a tBu group or a trimethylsilyl group.

Further, L may also be a group represented by the following Formula (G1).

The compound (1) having the group (G1) tends to have a low melting point.

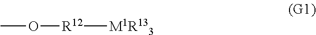

where
R¹² is an alkylene group, (Si(CH₃)₂O)ₘ₃, or a combination of an alkylene group and (Si(CH₃)₂O)ₘ₃,
M¹ is a carbon atom or a silicon atom,
R¹³ is each independently a hydrogen atom or an alkyl group which may have a branch, and each of at least two of R¹³ is an alkyl group which may have a branch, and
m3 is an integer from 1 to 12.

The alkylene group in R¹² may be a straight chain or may have a branch. Specific examples of R¹² include those similar to those of R². In particular, R¹² is preferably a linear alkylene group.

The alkyl group in R¹³ may be a straight chain or may have a branch. Specific examples of R¹³ include —CH₃, —CH₂CH₃, —CH₂CH₂CH₃, —CH(CH₃)CH₂C(CH₃)₃, and —CH₂CH₂CH(CH₃)CH₂C(CH₃)₃.

Examples of the combination of an alkylene group and (Si(CH₃)₂O)ₘ₃ in R¹² include —R¹⁴—(Si(CH₃)₂O)ₘ₃—, —(Si(CH₃)₂O)ₘ₃—R¹⁵— and —R¹⁴—(Si(CH₃)₂O)ₘ₃—R¹⁵—. However, R¹⁴ and R¹⁵ are each independently an alkylene group, and their specific examples include those similar to those of R². In particular, they are preferably linear alkylene groups.

M¹R¹³₃ may be CH(CH₃)₂, C(CH₃)₃, or Si(CH₃)₃ because the melting point of the compound (1) is easily lowered.

Specific examples of the group (G1) include groups shown below.

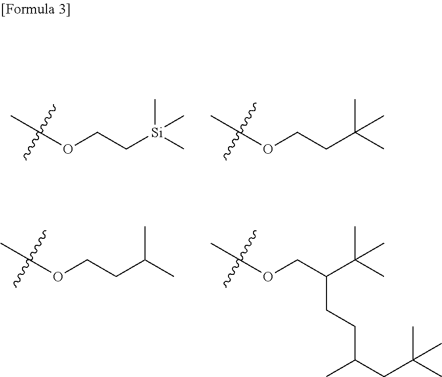

The group having a hydrolyzable group may be, for example, the group having a hydrolyzable group shown above as an example. The group having a hydrolyzable group is preferably —O-L^{A}-L^{B}. L^{A} is an alkylene group, and L^{B} is a hydrolyzable group. The number of carbon atoms of the alkylene group is preferably 1 to 10. The hydrolyzable group represented by L^{B} is synonymous with the above-described hydrolyzable group represented by L, and its preferred forms are also the same as those described above.

In particular, L is preferably an alkoxy group having 1 to 4 carbon atoms or a halogen atom in view of the ease of the manufacturing the compound. L is preferably an alkoxy group having 1 to 4 carbon atoms, and more preferably an ethoxy group or a methoxy group in view of the fact that outgassing during the application is small and because the storage stability of the compound becomes more excellent.

m2 is each independently an integer from 0 to 2.

m2 is preferably 0 or 1 and more preferably 0. The presence of a plurality of L makes the adhesive property of the surface-treated layer for the substrate stronger.

When m2 is 1 or less, a plurality of L present in one molecule may be the same as each other or different from each other. The plurality of L are preferably the same as each other in view of the availability of raw materials and the ease of the manufacturing of the compound. When m2 is 2, the plurality of R¹¹ present in one molecule may be the same as each other or different from each other. The plurality of R¹¹ are preferably the same as each other in view of the availability of raw materials and the ease of the manufacturing of the compound.

The compound (1) may be in a liquid state or in a solid state at 25° C. A surface treatment agent comprising a liquid medium or a surface treatment agent comprising a liquid medium (which will be described later) is preferably in a liquid state because its storage stability becomes excellent. The use of compound (1) which is in a liquid state at 25° C. suppresses the generation of foreign substances in the liquid composition. On the other hand, in order to improve the storage stability of the compound (1) itself, the compound (1) is preferably in a solid state at 25° C.

Examples of the means for making the compound (1) liquid at 25° C. include providing a branched chain at R¹ and/or T¹ of the compound (1); specifically, L being a group (G1), and setting m1 in R¹ to 2 to 3.

Note that whether the compound (1) is in a liquid state is determined by a test described below.

Two test tubes (a) and (b) marked with two mark lines A and B described below are prepared, and a sample is fed to the height of the mark line A in each of the test tubes. One test tube (a) is tightly closed with a rubber stopper, and the other test tube (b) is tightly closed with a rubber stopper through which a bar-shaped thermometer is inserted. The test tubes (a) and (b) are gently placed upright in a constant temperature water bath in such a manner that the mark lines B are under the water surface, and held for 10 minutes after the temperature measured by the thermometer of the test tube (b) reaches a target temperature. 10 minutes after that, the test tube (a) is taken out in the upright state and then immediately laid down horizontally on a table. The time from when the test tube (a) is laid down horizontally until the tip of the liquid surface of the sample passes mark line B is measured. When the measured time is within 90 seconds, it is determined that the sample is in a liquid state.

Note that as each of the test tubes, one having an inner diameter of 30 mm and a height of 120 mm, and having the mark line A at a height of 55 mm from the bottom of the tube and the mark line B at a height of 85 mm from the bottom of the tube is used.

Examples of the compound (1) include compounds shown below. However, it is not limited to these compounds. Further, in the following formula, “1” is added to the carbon atom at the end on the R¹ side in L¹, and the number of carbons contained in L¹ (such as 13, 19, or 21) is added to the carbon atom at the end on the T¹ side in L¹.

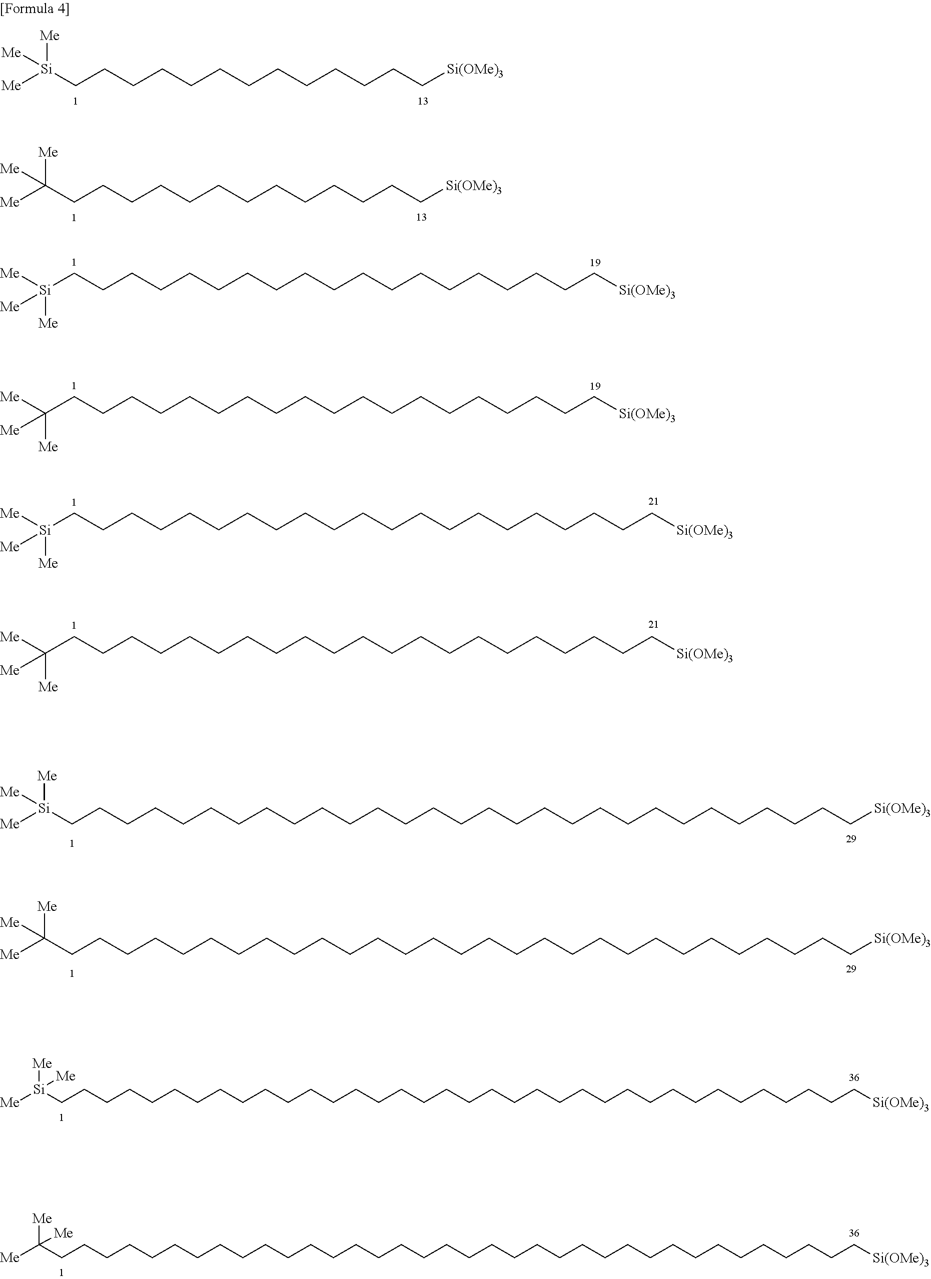

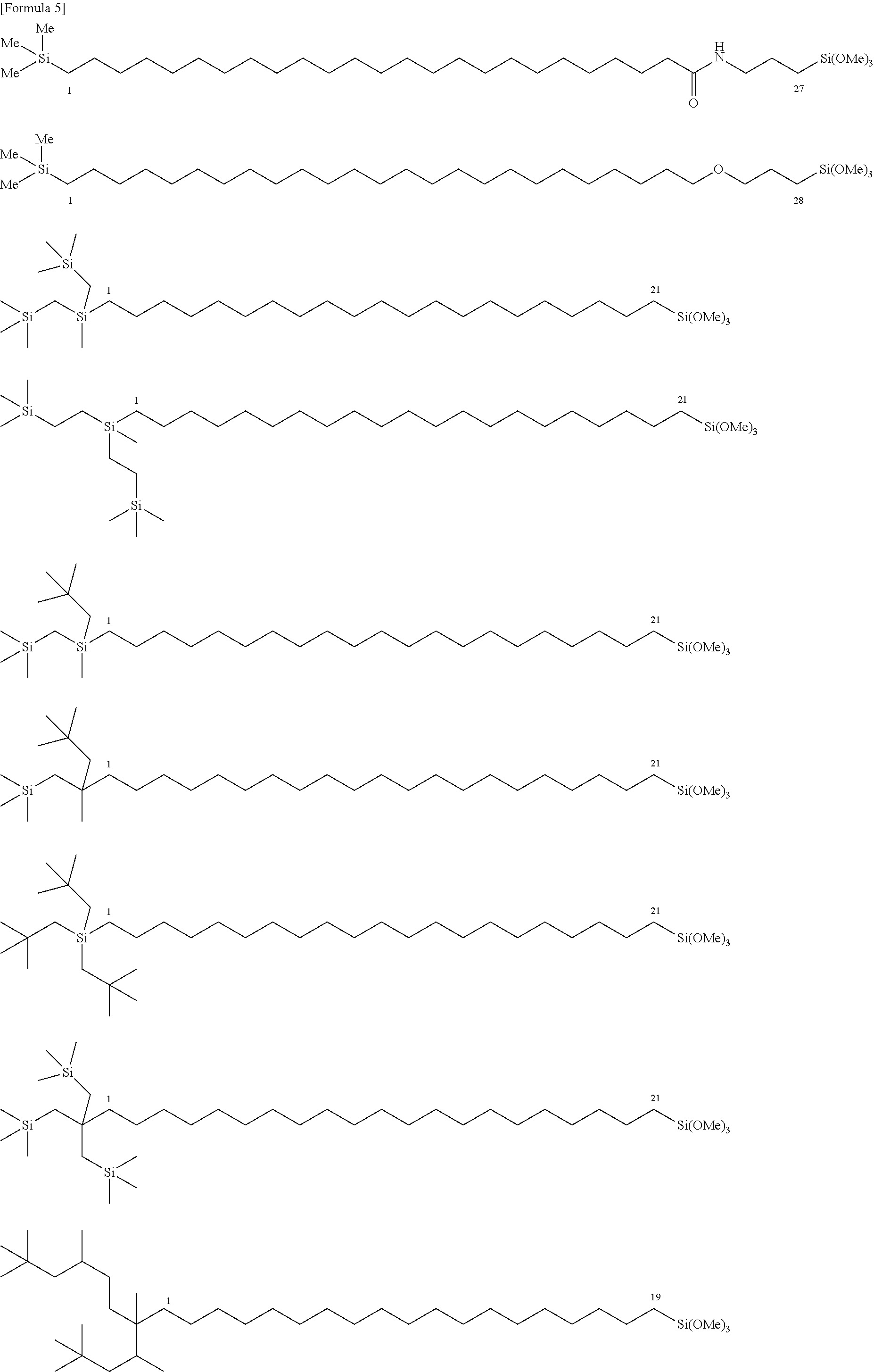

### <Method for Manufacturing Compound (1)>

The method for manufacturing compound (1) is not limited to any particular methods, and its examples include a method for manufacturing compound (1) by using a compound represented by the following Formula (4) as a raw material.

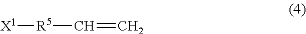

In Formula (4),
X¹ is a halogen atom, and is preferably Br or Cl, and
R⁵ is an alkylene group which may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.

Note that R⁵ is a part that constitutes a part of L¹ after the synthesis of the compound (1), and its number of carbon atoms is adjusted as appropriate while taking the desired number of carbon atoms of L¹ and the following reaction route into consideration.

Each step of the synthesis in a manufacturing method using compound (4) as a raw material will be described hereinafter. Examples of the synthesis routes include a route in which synthesis 1 and synthesis 3 described below are carried out in sequence, and a route in which synthesis 2 and synthesis 3 described below are carried out in sequence. Note that details of the solvent, the catalyst, the heating temperature, and the like used in each of the following syntheses can be selected as appropriate with reference to examples described later.

### (Synthesis 1)

The synthesis 1 is a method in which R¹ is introduced into the X¹ side of the compound (4). Examples of the synthesis 1 include a method in which the aforementioned compound (4) is reacted with a compound represented by the following Formula (5), and a compound represented by the following Formula (6) is thereby obtained.

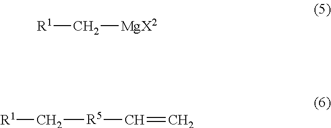

In Formulas (5) and (6),
R¹ is similar to that in the above-shown Formula (1),
X² is a halogen atom, and is preferably Br or Cl, and
R⁵ is similar to that in the above-shown Formula (4).

The above-described reaction 1 is a reaction for introducing R¹ into the X¹ side of the compound (4).

### (Synthesis 2)

The synthesis 2 is a method in which R¹ is introduced into the double-bond side of the compound (4). The synthesis 2 is carried out by combining, for example, the following syntheses 2-1 to 2-3.

### Synthesis 2-1

For example, a compound represented by the following Formula (8) is obtained by reacting the above-described compound (4) with a compound represented by the following Formula (7).

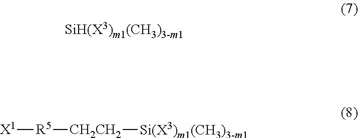

In Formulas (7) and (8),
X³ is a halogen atom, and is preferably Br or Cl,
m1 is similar to that in the above-shown Formula (1), and
X¹ and R⁵ are similar to those in the above-shown Formula (4).

### Synthesis 2-2

When X³ is present in the above-described compound (8), the compound represented by the following Formula (10) is obtained by reacting X³ with a compound represented by the following Formula (9).

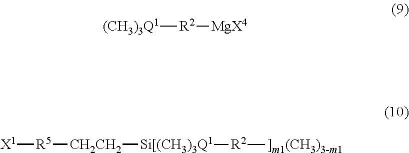

In Formulas (9) and (10),
X⁴ is a halogen atom, and is preferably Br or Cl,
Q¹, R², and m1 are similar to those in the above-shown Formula (1), and
X¹ and R⁵ are similar to those in the above-shown Formula (4).

Note that Si[(CH₃)₃Q¹-R²-]ₘ₁(CH₃)₃₋ₘ₁ corresponds to R¹ in Formula (1).

### Synthesis 2-3

When m1 is 0, the following compound (12) is obtained by reacting the above-described compound (8) or (10) with the above-described compound (11).

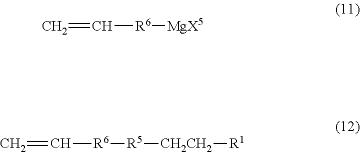

In Formulas (11) and (12),
X⁵ is a halogen atom, and is preferably Br or Cl,
R⁵ is similar to that in the above-shown Formula (4),
R⁶ is an alkylene group which may have one or more groups, selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms, and R¹ is similar to that in the above-shown Formula (1).

Note that when the compound (8) is used, R¹ is Si(CH₃)₃. Further, when the compound (10) is used, R¹ is Si[(CH₃)₃Q¹-R²-]ₘ₁(CH₃)₃₋ₘ₁.

When each of CH₂—R⁵ of the above-described compound (6) and R⁶-R⁵—CH₂CH₂ of the above-described compound (12) is L², the compound (6) and the compound (12) are both represented by the following common Formula (13). The compound (13) is a precursor compound suitable for manufacturing the compound (1).

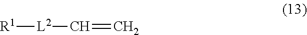

In Formula (13),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3, and
L² is an alkylene group having 11 or more carbon atoms, and may have one or more bonds selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.

Note that L²-CH═CH₂ is a part which becomes L¹ of the compound (1) by the following synthesis 3.

### (Synthesis 3)

The synthesis 3 is a method in which a reactive silyl group is introduced into the double bond of the compound (13). For example, the above-described compound (1) is obtained by reacting the compound (13) with a compound represented by the following Formula (14).

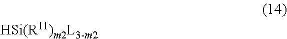

The symbols in Formula (14) are similar to those in the above-shown Formula (1).

### [Composition]

It is sufficient if a composition according to the present disclosure contains a compound according to the present disclosure, and its components other than the compound according to the present disclosure are not limited to any particular components. The composition according to the present disclosure preferably contains a compound according to the present disclosure and a liquid medium. In the case where the composition according to the present disclosure contains a liquid medium, it is sufficient if the liquid medium is in a liquid state. That is, the liquid medium may be a solution or a dispersion liquid.

It is sufficient if the composition according to the present disclosure contains a compound according to the present disclosure, and the composition may also contain impurities such as by-products generated in the process for manufacturing the compound according to the present disclosure.

The content of the compound according to the present disclosure is preferably 0.001 to 40 mass %, more preferably 0.01 to 20 mass %, and still more preferably 0.1 to 10 mass % based on the total amount of the composition according to the present disclosure. In the case where the composition according to the present disclosure is used in a wet coating method, the content of the compound according to the present disclosure is preferably 0.01 to 10 mass %, more preferably 0.02 to 5 mass %, still more preferably 0.03 to 3 mass %, and particularly preferably 0.05 to 2 mass % based on the total amount of the composition according to the present disclosure.

The composition according to the present disclosure may contain only one type of liquid medium, or may contain two or more types of liquid mediums.

The liquid medium is preferably an organic solvent.

Examples of organic solvents include compounds consisting solely of hydrogen atoms and carbon atoms, and compounds consisting solely of hydrogen atoms, carbon atoms, and oxygen atoms. Specifically, examples include hydrocarbon-based organic solvents, ketone-based organic solvents, ether-based organic solvents, ester-based organic solvents, glycol-based organic solvents, and alcohol-based organic solvents. Among them, the organic solvent is preferably a hydrocarbon-based organic solvent or an ester-based organic solvent.

Specific examples of hydrocarbon-based organic solvents include pentane, hexane, heptane, octane, hexadecane, isohexane, isooctane, isononane, cycloheptane, cyclohexane, bicyclohexyl, benzene, toluene, ethylbenzene, o-xylene, m-xylene, p-xylene, o-diethylbenzene, m-diethylbenzene, p-diethylbenzene, n-butylbenzene, sec-butylbenzene, and tert-butylbenzene.

Specific examples of ketone-based organic solvents include acetone, methyl ethyl ketone, methyl isobutyl ketone, diisobutyl ketone, cyclohexanone, 2-heptanone, 4-heptanone, 3,5,5-trimethyl-2-cyclohexene-1-one, 3,3,5-trimethylcyclohexanone, and isophorone.

Specific examples of ether-based organic solvents include diethyl ether, cyclopentyl methyl ether, tetrahydrofuran, and 1,4-dioxane.

Specific examples of ester-based organic solvents include methyl acetate, ethyl acetate, propyl acetate, isopropyl acetate, butyl acetate, isobutyl acetate, tert-butyl acetate, amyl acetate, isoamyl acetate, ethyl 3-ethoxypropionate, ethyl lactate ethylene glycol monobutyl ether acetate, diethylene glycol monobutyl ether acetate, propylene glycol monomethyl ether acetate, dipropylene glycol methyl ether acetate, 3-methoxy-3-methylbutyl acetate, 3-methoxybutyl acetate, propylene glycol monomethyl acetate, propylene glycol dimethyl acetate, ethylene glycol monoethyl ether acetate, ethylene glycol monomethyl ether acetate, diethylene glycol monoethyl ether acetate, cyclohexanol acetate, propylene glycol diacetate, propylene glycol monomethyl ether acetate, propylene glycol monomethyl ether propionate, propylene glycol monoethyl ether acetate, propylene glycol monobutyl ether acetate, propylene glycol monopropyl ether acetate, dipropylene glycol-methyl ether acetate, 1,3-butylene glycol diacetate, 1,4-butanediol diacetate, 1,3-butylene glycol diacetate, 1,6-hexanediol diacetate, γ-butyrolactone, triacetin, and 2,2,4-trimethyl-1,3-pentanediol monoisobutyrate.

Specific examples of glycol-based organic solvents include ethylene glycol, ethylene glycol monobutyl ether, diethylene glycol monobutyl ether, triethylene glycol monobutyl ether, tetraethylene glycol monobutyl ether, ethylene glycol monohexyl ether, diethylene glycol monohexyl ether, ethylene glycol mono-2-ethylhexyl ether, diethylene glycol mono-2-ethylhexyl ether, propylene glycol monomethyl ether, propylene glycol monoethyl ether, propylene glycol monobutyl ether, propylene glycol monopropyl ether, ethylene glycol monoisopropyl ether, ethylene glycol monoethyl ether, ethylene glycol mono tert-butyl ether, ethylene glycol monopropyl ether, ethylene glycol monomethyl ether, diethylene glycol monoisopropyl ether, diethylene glycol monomethyl ether, dipropylene glycol monoethyl ether, dipropylene glycol monobutyl ether, dipropylene glycol monopropyl ether, dipropylene glycol monomethyl ether, dipropylene glycol monopropyl ether, dipropylene glycol monomethyl ether, tripropylene glycol monobutyl ether tripropylene glycol monomethyl ether, propylene glycol monophenyl ether, 1,3-butylene glycol, propylene glycol n-propyl ether, propylene glycol n-butyl ether, diethylene glycol monoethyl ether, dipropylene glycol n-propyl ether, dipropylene glycol n-butyl ether, tripropylene glycol methyl ether, tripropylene glycol n-butyl ether, ethylene glycol dimethyl ether, ethylene glycol diethyl ether, diethylene glycol diethyl ether, diethylene glycol dimethyl ether, diethylene glycol dimethyl ether, dipropylene glycol dimethyl ether, diethylene glycol dibutyl ether, tetraethylene glycol dimethyl ether, triethylene glycol dimethyl ether, and polyethylene glycol dimethyl ether.

Specific examples of alcohol-based organic solvents include methanol, ethanol, 1-propanol, isopropyl alcohol, n-butanol, diacetone alcohol, isobutanol, sec-butanol, tert-butanol, pentanol, 3-methyl-1,3-butanediol, 1,3-butanediol, 1,3-butylene glycol, octanediol, 2,4-diethylpentanediol, butylethylpropanediol, 2-methyl-1,3-propanediol, 4-hydroxy-4-methyl-2-pentanone, 2-ethyl-1-hexanol, 3,5,5-trimethyl-1-hexanol, isodecanol, isotridecanol, 3-methoxy-3-methyl-1-butanol, 2-methoxybutanol, 3-methoxybutanol, cyclohexanol, furfuryl alcohol, tetrahydrofurfuryl alcohol, benzyl alcohol, and methylcyclohexanol.

Further, examples of organic solvents include halogen-based organic solvents, nitrogen-containing compounds, sulfur-containing compounds, siloxane compounds, and fluorine-containing organic solvents.

Specific examples of halogen-based organic solvents include dichloromethane, chloroform, carbon tetrachloride, dichloroethane, chlorobenzene, o-chlorotoluene, m-chlorotoluene, p-chlorotoluene, m-dichlorobenzene, and 1,2,3-trichloropropane.

Examples of nitrogen-containing compounds include nitrobenzene, acetonitrile, benzonitrile, N,N-dimethylformamide, N,N-dimethylacetamide, N-methylpyrrolidone, and 1,3-dimethyl-2-imidazolidinone.

Examples of sulfur-containing compounds include carbon disulfide and dimethyl sulfoxide.

Examples of siloxane compounds include hexamethyldisiloxane, hexaethyldisiloxane, octamethyltrisiloxane, octaethyltrisiloxane, hexamethylcyclotrisiloxane, hexamethylcyclotrisiloxane, octamethylcyclotetrasiloxane, octaethylcyclotetrasiloxane, and decamethyltetrasiloxane.

Examples of fluorine-containing organic solvents include polyfluoro aromatic hydrocarbons (e.g., 1,3-bis(trifluoromethyl) benzene); polyfluoro aliphatic hydrocarbons (e.g., C₆F₁₃CH₂CH₃ (e.g., Asahikrin (Registered Trademark) AC-6000 manufactured by AGC Inc.), 1,1,2,2,3,3,4-heptafluorocyclopentane (e.g., Zeorora (Registered Trademark) H manufactured by Zeon Corporation); hydrofluoroethers (HFE) (e.g., alkyl perfluoroalkyl ethers (perfluoroalkyl group and alkyl group may be linear chain or branched) such as perfluoropropyl methyl ether (C₃F₇OCH₃) (e.g., Novec (Registered Trademark) 7000 manufactured by 3M Japan Limited), perfluorobutyl methyl ether (C₄F₉OCH₃) (e.g., Novec (Registered Trademark) 7100 manufactured by 3M Japan Limited), perfluorobutyl ethyl ether (C₄F₉OC₂H₅) (e.g., Novec (Registered Trademark) 7200 manufactured by 3M Japan Limited), and perfluorohexyl methyl ether (C₂F₅CF(OCH₃)C₃F₇) (e.g., Novec (Registered Trademark) 7300 manufactured by 3M Japan Limited), and CF₃CH₂OCF₂CHF₂ (e.g., Asahikrin (Registered Trademark) AE-3000 manufactured by AGC Inc.); and hydrofluoroolefins (HFO) (e.g., 1-chloro-2,3,3-trifluoro-1-propene (HCFO-1233yd) (e.g., Amolea (Registered Trademark) AS-300 manufactured by AGC Inc.)).

The content of the liquid medium is preferably 60 to 99.999 wt. %, more preferably 80 to 99.99 wt. %, and still more preferably 90 to 99.9 wt. % based on the total amount of the composition according to the present disclosure. In the case where the composition according to the present disclosure is used in a wet coating method, the content of the liquid medium is preferably 90 to 99.99 wt. %, more preferably 95 to 99.98 wt. %, still more preferably 97 to 99.97 wt. %, and particularly preferably 98 to 99.95 wt. % based on the total amount of the composition according to the present disclosure.

In addition to the compound according to the present disclosure and the liquid medium, the composition according to the present disclosure may contain other components in a range in which the effects of the present disclosure are not impaired.

Examples of other components include additives, specifically, catalysts, such as acid catalysts and base catalysts, which accelerate the hydrolysis and the condensation reaction of a reactive silyl group.

For example, an arbitrary suitable acid or base, a transition metal (e.g., Ti, Ni, Sn, Zr, Al or B), a sulfur-containing compound having a non-covalent electron pair in the molecular structure, a nitrogen-containing compound (e.g., a sulfoxide compound, an aliphatic amine compound, an aromatic amine compound, a phosphoric acid amide compound, an amide compound, and a urea compound) can be used as the catalyst.

Examples of acid catalysts include acetic acid, formic acid, trifluoroacetic acid, hydrochloric acid, nitric acid, sulfuric acid, phosphoric acid, sulfonic acid, methanesulfonic acid, and p-toluenesulfonic acid.

Further, examples of base catalysts include ammonia, sodium hydroxide, and potassium hydroxide; and organic amines such as triethylamine and diethylamine.

Further, examples of other components also include metal compounds having hydrolyzable groups (hereinafter also referred to as “specific metal compounds”). When the composition according to the present disclosure contains a specific metal compound, the sliding property and the antifouling property of the surface-treated layer can be further improved. Examples of specific metal compounds include a metal compound represented by one of Formulas (M1) to (M3).

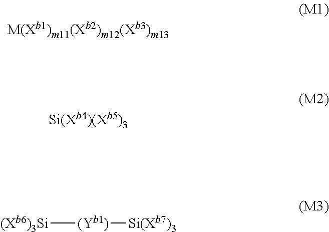

In Formula (M1),
M is a trivalent or tetravalent metal atom,
X^{b1} is each independently a hydrolyzable group,
X^{b2} is each independently a siloxane skeleton-containing group,
X^{b3} is each independently a hydrocarbon chain-containing group,
m11 is an integer from 2 to 4,
m12 and m13 are each independently an integer from 0 to 2, and
when M is a trivalent metal atom, m11+m12+m13 is 3, whereas when M is a tetravalent metal atom, m11+m12+m13 is 4.
In Formula (M2).
X^{b4} is a hydrolyzable silane oligomer residue, and
X^{b5} is each independently a hydrolyzable group or an alkyl group having 1 to 4 carbon atoms.
In Formula (M3),
X^{b6} and X^{b7} are each independently a hydrolyzable group or a hydroxyl group, and
Y^{b1} is a divalent organic group.

The metal represented by M in Formula (M1) includes semimetals such as Si and Ge. M is preferably a trivalent metal or a tetravalent metal, more preferably Al, Fe, In, Hf, Si, Ti, Sn, or Zr, still more preferably Al, Si, Ti, or Zr, and particularly preferably Si.

The hydrolyzable group represented by X^{b1} in Formula (M1) is synonymous with the hydrolyzable group represented by L in the above-shown Formula (1), and its preferred forms are also the same as those described above.

The siloxane skeleton-containing group represented by X^{b2} has a siloxane unit (—Si—O—), and may be either a linear chain or a branched chain. The siloxane unit is preferably a dialkylsilyloxy group, and examples include a dimethylsilyloxy group and a dimethylsilyloxy group. The number of repetitions of the siloxane unit in the siloxane skeleton-containing group is 1 or greater, preferably 1 to 5, more preferably 1 to 4, and still more preferably 1 to 3.

The siloxane skeleton-containing group may contain a divalent hydrocarbon group in a part of the siloxane skeleton. Specifically, an oxygen atom in a part of the siloxane skeleton may be replaced with a divalent hydrocarbon group. Examples of the aforementioned divalent hydrocarbon group include alkylene groups such as a methylene group, an ethylene group, a propylene group, and a butylene group.

A hydrolyzable group, a hydrocarbon group (preferably an alkyl group), or the like may be bonded to a silicon atom at the end of the siloxane skeleton-containing group.

The number of atoms of the siloxane skeleton-containing group is preferably 100 or less, more preferably 50 or less, and still more preferably 30 or less. The lower limit of the number of atoms is preferably 10 or more.

The siloxane skeleton-containing group is preferably a group represented by *—(O—Si(CH₃)₂)ₙCH₃, where n is an integer from 1 to 5 and * indicates a part bonded to an adjacent atom.

The hydrocarbon chain-containing group represented by X^{b3} may be a group consisting solely of a hydrocarbon chain or a group having an etheric oxygen atom between carbon atoms of a hydrocarbon chain. The hydrocarbon chain may be a linear chain or a branched chain, and is preferably a linear chain. The hydrocarbon chain may be a saturated hydrocarbon chain or an unsaturated hydrocarbon chain, and is preferably a saturated hydrocarbon chain. The number of carbon atoms of the hydrocarbon chain-containing group is preferably 1 to 3, more preferably 1 to 2, and still more preferably 1. The hydrocarbon chain-containing group is preferably an alkyl group, and more preferably a methyl group, an ethyl group, or a propyl group.

m1 is preferably 3 or 4.

The compound represented by Formula (M1) is preferably a compound represented by one of Formulas (M1-1) to (M1-5) in which M is Si, and more preferably a compound represented by Formula (M1-1). The compound represented by Formula (M1-1) is preferably tetraethoxysilane, tetramethoxysilane, or triethoxymethylsilane.

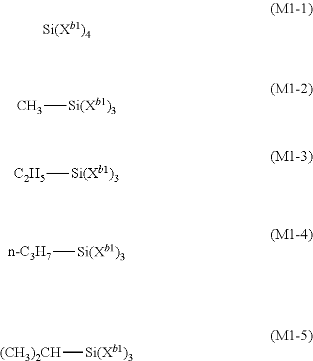

In Formula (M2), the number of silicon atoms contained in the hydrolyzable silane oligomer residue represented by X^{b4} is preferably 3 or greater, more preferably 5 or greater, and still more preferably 7 or greater. The number of silicon atoms is preferably 15 or less, more preferably 13 or less, and still more preferably 10 or less.

The hydrolyzable silane oligomer residue may have an alkoxy group that is bonded to a silicon atom. Examples of the aforementioned alkoxy group include a methoxy group, an ethoxy group, a propoxy group, and a butoxy group. Further, the alkoxy group is preferably a methoxy group or an ethoxy group. The hydrolyzable silane oligomer residue may have one or two or more types of alkoxy groups, and preferably has one type of alkoxy group.

Examples of the hydrolyzable silane oligomer residue include (C₂H₅O)₃Si—(OSi(OC₂H₅)₂)₄O—*. Note that * indicates a part bonded to an adjacent atom.

Examples of the hydrolyzable group represented by X^{b5} in Formula (M2) include those similar to the hydrolyzable group represented by L in the above-shown Formula (1-1), a cyano group, a hydrogen atom, and an allyl group. Further, the hydrolyzable group is preferably an alkoxy group or an isocyanato group. The alkoxy group is preferably an alkoxy group having 1 to 4 carbon atoms.

X^{b5} is preferably a hydrolyzable group.

Examples of the compound represented by Formula (M2) include (H₅C₂O)₃—Si—(OSi(OC₂H₅)₂)₄OC₂H₅.

The compound represented by Formula (M3) is a compound having reactive silyl groups at both ends of a divalent organic group, i.e., is bissilane.

Examples of the hydrolyzable groups represented by X^{b6} and X^{b7} in Formula (M3) include an alkoxy group, an acyloxy group, a ketoxime group, an alkenyloxy group, an amino group, an aminoxy group, an amide group, an isocyanato group, and a halogen atom. Further, the hydrolyzable group is preferably an alkoxy group or an isocyanato group. The alkoxy group is preferably an alkoxy group having 1 to 4 carbon atoms, and still more preferably a methoxy group or an ethoxy group.

In Formula (M3), X^{b6} and X^{b7} may be the same groups as each other or groups different from each other. In view of the availability, X^{b6} and X^{b7} are preferably the same groups as each other.

In Formula (M3), Y^{b1} is a divalent organic group connecting reactive silyl groups at both ends. The number of carbon atoms of Y^{b1} of the divalent organic group is preferably 1 to 8 and more preferably 1 to 3.

Examples of Y^{b1} include an alkylene group, a phenylene group, and an alkylene group having an etheric oxygen atom between carbon atoms. Examples include —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(CH₃)₂CH₂—, —C(CH₃)₂CH₂CH₂C(CH₃)₂—, —CH₂CH₂OCH₂CH₂—, —CH₂CH₂CH₂OCH₂CH₂CH₂—, —CH(CH₃)CH₂OCH₂CH(CH₃)—, and —C₆H₄—.

Examples of the compound represented by Formula (M3) include (CH₃O)₃Si(CH₂)₂Si(OCH₃)₃, (C₂H₅O)₃Si(CH₂)₂Si(OC₂H₅)₃, (OCN)₃Si(CH₂)₂Si(NCO)₃, Cl₃Si(CH₂)₂SiCl₃, (CH₃₀)₃Si(CH₂)₆Si(OCH₃)₃, and (C₂H₅O)₃Si(CH₂)₆Si(OC₂H₅)₃.

The content of other components which may be contained in the composition according to the present disclosure is preferably 10 mass % or less and more preferably 1 mass % or less based on the total amount of the composition according to the present disclosure. When the composition according to the present disclosure contains a specific metal compound, the content of the specific metal compound is preferably 0.01 to 30 wt. %, more preferably 0.01 to 10 wt. %, and still more preferably 0.05 to 5 wt. % based on the total amount of the composition according to the present disclosure.

The total content (hereinafter also referred to as “solid content concentration”) of the compound according to the present disclosure and the other components is preferably 0.001 to 40 wt. %, more preferably 0.01 to 20 wt. %, and still more preferably 0.1 to 10 wt. % based on the total amount of the composition according to the present disclosure. The solid content concentration of the composition according to the present disclosure is a value calculated from the mass of the composition before being heated and the mass thereof after being heated in a convection-type dryer at 120° C. for 4 hours.

Since the composition according to the present disclosure contains a liquid medium, it is useful for use in which the composition is used for coating, and can be used as a coating liquid.

Examples of the other components include compounds represented by the following Formula (15).

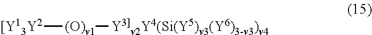

In Formula (15), Y² is Si, Sn, or Ge,
Y¹ is each independently a hydrocarbon group or a trialkylsilyloxy group,
v1 is 0 or 1,
Y³ is each independently an alkylene chain or a polyalkylene oxide chain, or a combination of an alkylene chain and a divalent polysiloxane residue,
Y⁴ is a single bond or a linking group having a valence of (v2+v4),
Y⁵ is each independently a hydrocarbon group,
Y⁶ is each independently a hydrolyzable group or a hydroxyl group,
v3 is each independently an integer from 0 to 2, and
v2 and v4 are each independently an integer from 1 or greater.

The compound (15) is preferably one in which Y³ is an alkylene chain or a polyalkylene oxide chain.

Specific examples of the compound (15) include compounds shown below. γ in the formula is preferably 9 to 50, more preferably 11 to 30, and particularly preferably 11 to 25.

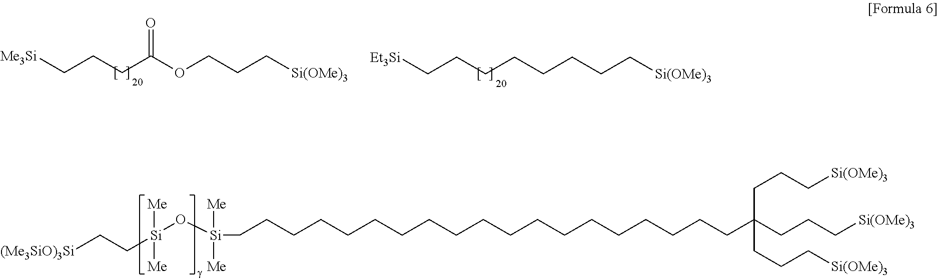

When the other component in the surface treatment agent disclosed herein is the compound (15), the content of the compound (15) is preferably 50 mass % or less and more preferably 40 mass % or less.

### [Surface Treatment Agent]

In an embodiment, a surface treatment agent according to the present disclosure contains a compound according to the present disclosure. Further, a surface treatment agent according to the present disclosure may contain a compound according to the present disclosure and a liquid medium. A surface treatment agent according to the present disclosure may be a composition according to the present disclosure. Preferred embodiments of the liquid medium contained in the surface treatment agent according to the present disclosure are similar to those of the liquid medium contained in the composition according to the present disclosure.

### [Article]

In an embodiment, an article according to the present disclosure includes a substrate and a surface-treated layer which is disposed on the substrate and of which the surface is treated with a surface treatment agent according to the present disclosure.

The surface-treated layer may be formed on a part of the surface of the substrate or over the entire surface of the substrate. The surface-treated layer may be spread in the form of a film on the surface of the substrate or scattered in the form of dots.

In the surface-treated layer, the compound according to the present disclosure is contained therein in a state in which the hydrolysis of some or all of reactive silyl groups has progressed and the dehydration condensation reaction of silanol groups has progressed.

The thickness of the surface-treated layer is preferably 1 to 100 nm and more preferably 1 to 50 nm. When the thickness of the surface-treated layer is 1 nm or larger, the effects of the surface treatment are likely to be obtained satisfactorily. When the thickness of the surface-treated layer is 100 nm or smaller, the usage efficiency is high. The thickness of the surface-treated layer can be calculated from the oscillation cycle of the interference pattern of the reflected X-ray, which is obtained by an X-ray reflectivity method using an X-ray diffractometer for thin film analysis (Product name: ATX-G, manufactured by Rigaku Corporation).

The type of the substrate is not limited to any particular types, and examples include a substrate which is required to have water repellency. Examples of the substrate include a substrate which may be used while touching it with another article (e.g., a stylus) or a human hand or fingers; a substrate which may be held by a human hand or fingers during the operation; and a substrate which may be placed on another article (e.g., a mounting table).

Examples of the materials of the substrate include metals, resins, glass, sapphire, ceramics, semiconductors, stones, fibers, nonwoven fabrics, paper, wood, fur, natural leather, artificial leather, porcelain, and composite materials thereof. The glass may be one that is chemically reinforced.

Examples of substrates including building materials, decorative building materials, interior goods, transportation apparatuses (e.g., automobiles), signboards, bulletin boards, drinking containers, tableware, water tanks, ornamental apparatuses (e.g., frames and boxes), laboratory apparatuses, furniture, textile products, and packaging containers; glass or resins used for art, sports, games and the like; glass or resins used for exterior parts (excluding display parts) of apparatuses such as mobile phones (e.g., smartphones), mobile information terminals, game machines, and remote controllers. The shape of the substrate may be plate-like or film-like.

The substrate is preferably a substrate for a touch panel, a substrate for a display, or a lens for eyeglasses, and particularly preferably a substrate for a touch panel. The material of a substrate for a touch panel is preferably glass or a transparent resin.

The substrate may be a substrate of which a surface treatment such as a corona discharge treatment, a plasma treatment, a plasma graft polymerization treatment, or the like has been performed on one or both of the surfaces. The substrate subjected to the surface treatment has a more excellent adhesive property for the surface-treated layer, and the abrasion resistance of the surface-treated layer is further improved. Therefore, it is preferred to perform a surface treatment on the surface of the substrate on the side which is brought into contact with the surface-treated layer. Further, in the case where an underlayer (which will be described later) is provided, the substrate subjected to the surface treatment has a more excellent adhesive property for the underlayer, and the abrasion resistance of the surface-treated layer is further improved. Therefore, in the case where an underlayer is provided, it is preferred to perform a surface treatment on the surface of the substrate on the side which is brought into contact with the underlayer.

The surface-treated layer may be directly disposed on the surface of the substrate, or an underlayer may be provided between the substrate and the surface-treated layer. In order to further improve the water repellency and the abrasion resistance of the surface-treated layer, an article according to the present disclosure preferably includes a substrate, an underlayer disposed on the substrate, and a surface-treated layer which is disposed on the underlayer and of which the surface is treated with a surface treatment agent according to the present disclosure.

The underlayer is preferably a layer comprising an oxide comprising silicon and at least one specific element selected from the group consisting of Group 1 elements, Group 2 elements, Group 4 elements, Group 5 elements, Group 13 elements, and Group 15 elements in the periodic table.

Group 1 elements in the periodic table (hereinafter also referred to simply as “Group 1 elements”) refer to lithium, sodium, potassium, rubidium, and cesium. Group 1 elements are preferably lithium, sodium, and potassium, and more preferably sodium and potassium because the surface-treated layer can be formed more uniformly on the underlayer without defects or because variations in the composition of the underlayer among samples are more suppressed. The underlayer may contain two or more Group 1 elements.

Group 2 elements in the periodic table (hereinafter also referred to simply as “Group 2 elements”) refer to beryllium, magnesium, calcium, strontium, and barium. Group 2 elements are preferably magnesium, calcium, and barium, and more preferably magnesium and calcium because the surface-treated layer can be formed more uniformly on the underlayer without defects or because variations in the composition of the underlayer among samples are more suppressed. The underlayer may contain two or more Group 2 elements.

Group 4 elements in the periodic table (hereinafter also referred to simply as “Group 4 elements”) refer to titanium, zirconium, and hafnium. Group 4 elements are preferably titanium and zirconium, and more preferably titanium because the surface-treated layer can be formed more uniformly on the underlayer without defects or because variations in the composition of the underlayer among samples are more suppressed. The underlayer may contain two or more Group 4 elements.

Group 5 elements in the periodic table (hereinafter also referred to simply as “Group 5 elements”) refer to vanadium, niobium, and tantalum. Group 5 elements are particularly preferably vanadium because the abrasion resistance of the surface-treated layer becomes more excellent. The underlayer may contain two or more Group 5 elements.

Group 13 elements in the periodic table (hereinafter also referred to simply as “Group 13 elements”) refer to boron, aluminum, gallium, and indium. Group 13 elements are preferably boron, aluminum, and gallium, and more preferably boron and aluminum because the surface-treated layer can be formed more uniformly on the underlayer without defects or because variations in the composition of the underlayer among samples are more suppressed. The underlayer may contain two or more Group 13 elements.

Group 15 elements in the periodic table (hereinafter also referred to simply as “Group 15 elements”) refer to nitrogen, phosphorus, arsenic, antimony, and bismuth. Group 15 elements are preferably phosphorus, antimony, and bismuth, and more preferably phosphorus and bismuth because the surface-treated layer can be formed more uniformly on the underlayer without defects or because variations in the composition of the underlayer among samples are more suppressed. The underlayer may contain two or more Group 15 elements.

The specific element contained in the underlayer is preferably a Group 1 element, a Group 2 element, and a Group 13 element, more preferably a Group 1 element and a Group 2 element, and still more preferably a Group 1 element because the abrasion resistance of the surface-treated layer becomes more excellent.

Only one element or two or more elements may be contained as the specific element(s).

The oxide contained in the underlayer may be a mixture of oxides each comprising only one of the aforementioned elements (silicon and specific element) (e.g., a mixture of silicon oxide and an oxide of a specific element), a composite oxide comprising two or more of the aforementioned elements, or a mixture of an oxide comprising only one of the aforementioned elements and a composite oxide.

The ratio of the total molarity of the specific element in the underlayer to the molarity of silicon in the underlayer (specific element/silicon) is preferably 0.02 to 2.90, more preferably 0.10 to 2.00, and still more preferably 0.20 to 1.80 because the abrasion resistance of the surface-treated layer becomes more excellent.

The molarity (mol %) of each element in the underlayer can be measured, for example, by a depth-direction analysis by X-ray photoelectron spectroscopy (XPS) using ion sputtering.

The underlayer may be a single layer or may consist of a plurality of layer. The underlayer may have an uneven surface(s).

The thickness of the underlayer is preferably 1 to 100 nm, more preferably 1 to 50 nm, and still more preferably 2 to 20 nm. When the thickness of the underlayer is equal to or larger than the aforementioned lower limit value, the adhesive property of the surface-treated layer by the underlayer is further improved, so that the abrasion resistance of the surface-treated layer becomes more excellent. When the thickness of the underlayer is equal to or less than the aforementioned upper limit value, the abrasion resistance of the underlayer itself becomes excellent.

The thickness of the underlayer is measured by observing the cross section of the underlayer by a transmission electron microscope (TEM).

The underlayer can be formed, for example, by a vapor-deposition method using a vapor-deposition material or a wet coating method.

The vapor-deposition material used in the vapor-deposition method preferably contains silicon and an oxide comprising a specific element.

Specific examples of the form of the vapor-deposition material include a powder, a molten material, a sintered compact, granules, and a crushed material. Further, a molten material, a sintered compact, and granules are preferred in view of the handling property.

Note that the molten material refers to a solid material obtained by melting a powder of a vapor-deposition material at a high temperature, and then cooling and solidifying the molten vapor-deposition material. The sintered compact refers to a solid material obtained by firing a powder of a vapor-deposition material. Further, if necessary, instead of the powder of the vapor-deposition material, a molded compact formed by pressing the powder may be used. The granules refers to a solid material obtained by mixing and kneading a powder of a vapor-deposition material and a liquid medium (e.g., water or an organic solvent) and thereby obtaining particles thereof, and then drying the obtained particles.

The vapor-deposition material can be manufactured, for example, by the following methods.
A method in which a powder of a vapor-deposition material is obtained by mixing a powder of silicon oxide with a powder of an oxide of a specific element.
A method in which granules of a vapor-deposition material is obtained by, after obtaining particles by mixing and kneading a powder of the aforementioned vapor-deposition material and water, drying the obtained particles.
A method in which a sintered compact is obtained by drying a mixture obtained by mixing a powder comprising silicon (e.g., a powder made of silicon oxide, silica sand, or silica gel), a powder comprising a specific element (e.g., a powder of an oxide of a specific element, a carbonate, a sulfate, a nitrate, an oxalate, or a hydroxide), and water, and then firing the dried mixture or a molded article obtained by press-molding the dried mixture.
A method in which a molten material is obtained by melting a mixture obtained by mixing a powder comprising silicon (e.g., a powder made of silicon oxide, silica sand, or silica gel), a powder comprising a specific element (e.g., a powder of an oxide of a specific element, a carbonate, a sulfate, a nitrate, an oxalate, or a hydroxide), and water at a high temperature, and then cooling and solidifying the molten substance.

Specific examples of the vapor-deposition method using a vapor-deposition material include a vacuum vapor-deposition method. The vacuum vapor-deposition method is a method in which a vapor-deposition material is evaporated in a vacuum chamber and deposited on the surface of a substrate.

The temperature during the vapor-deposition (e.g., in the case where a vacuum deposition apparatus is used, the temperature of a boat in which the deposition material is disposed) is preferably 100 to 3,000° C. and more preferably 500 to 3,000° C.

The pressure during the vapor-deposition (e.g., in the case where a vacuum deposition apparatus is used, the pressure in a chamber in which the deposition material is disposed) is preferably 1 Pa or lower and more preferably 0.1 Pa or lower.

When an underlayer is formed by using a vapor-deposition material, one vapor-deposition material may be used, or two or more vapor-deposition materials comprising different elements may be used.

Specific examples of the method for evaporating a vapor-deposition material include a resistive heating method in which a vapor-deposition material is melted and evaporated on a resistive heating boat made of a high melting-point metal, and an electron gun method in which a vapor-deposition material is irradiated with an electron beam and thereby directly heated, so that its surface is melted and evaporated. As the method for evaporating a vapor-deposition material, the electron gun method is preferred because since the material can be locally heated, even a material having a high melting point can be evaporated, and since an area that is not irradiated with the electron beam is kept at a low temperature, there is no risk of reaction with the container nor risk of contamination by impurities.

As the method for evaporating a vapor-deposition material, a plurality of boats may be used, or the whole vapor-deposition materials may be put in a single boat. The vapor-deposition method may be co-vapor deposition or alternating vapor deposition. Specific examples include an example in which silica and a specific source are mixed and used in the same boat, an example in which co-vapor deposition is performed in a state in which silica and a specific element source put in separate boats, and an example in which alternating vapor deposition is performed in a similar manner, i.e., in a state in which silica and a specific element source are put in separate boats. The conditions for the vapor deposition, the order, and the like are selected as appropriate according to the structure of the underlayer.

In the wet coating method, it is preferred to form an underlayer on a substrate by a wet coating method using a coating liquid comprising a compound comprising silicon, a compound comprising a specific element, and a liquid medium.

Specific examples of silicon compounds include silicon oxide, silicic acid, partial condensate of silicic acid, alkoxysilane, and partial hydrolysis condensate of alkoxysilane.

Specific examples of compounds comprising a specific element include oxides of the specific element, alkoxides of the specific element, carbonates of the specific element, sulfates of the specific element, nitrates of the specific element, oxalates of the specific element, and hydroxides of the specific element.

Examples of liquid mediums include those similar to liquid mediums contained in the composition according to the present disclosure.

The content of the liquid medium is preferably 0.01 to 20 mass % and more preferably 0.1 to 10 mass % based on the total amount of the coating liquid used for the formation of the underlayer.

Specific examples of the wet coating method for forming an underlayer include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

After the substrate or the like is wet-coated with the coating liquid, the coating is preferably dried. The drying temperature of the coating is preferably 20 to 200° C. and more preferably 80 to 160° C.

The article according to the present disclosure may be an optical material including a surface-treated layer as the outermost layer.

Examples of preferred optical materials include a wide variety of optical materials in addition to optical materials related to displays or the like.

Examples of optical materials include a cathode ray tube (CRT; e.g., a computer monitor), a display such as a liquid crystal display, a plasma display, an organic EL display, an inorganic thin film EL dot matrix display, a rear projection-type display, a vacuum fluorescent display (VFD), and a field emission display (FED), a protective plate for such a display, and those in which an antireflection film treatment is performed on their surfaces.

The article according to the present disclosure is preferably an optical member. Examples of optical members include a car navigation system, a mobile phone, a smartphone, a digital camera, a digital video camera, a PDA, a portable audio player, a car audio system, a game apparatus, a lens for eyeglasses, a camera lens, a lens filter, sunglasses, a medical apparatus such as a gastroscope, a copying machine, a PC, a display (e.g., a liquid crystal display, an organic EL display, a plasma display, and a touch panel display), a touch panel, a protective film, and an antireflection film.

Further, examples of optical members include a front protective plate for a display such as a PDP and an LCD, an antireflection plate, a polarizing plate, and an antiglare plate; a disk surface of an optical disk such as a Blu-ray (Blu-ray (Registered Trademark)) disk, a DVD disk, a CD-R, and an MO; an optical fiber; and a display surface of a clock or a watch.

In particular, the article according to the present disclosure is preferably a display or a touch panel.

The article according to the present disclosure may be a medical apparatus or a medical material. Further, the article according to the present disclosure may also be an automobile interior or exterior member. Examples of exterior members include a window, a light cover, and an external camera cover. Examples of interior members include an instrument panel cover, a navigation system touch panel, and a decorative interior member.

When the article according to the present disclosure is an optical member, the material constituting the surface of the substrate is a member for an optical member, e.g., glass or transparent plastic. Further, when the article according to the present disclosure is an optical member, a functional layer such as a hard coat layer or an antireflection layer may be formed on the surface (outermost layer) of the substrate. The antireflection layer may be either a single-layer antireflection layer or a multi-layer antireflection layer.

Examples of inorganic substances that can be used for the antireflection layer include SiO₂, SiO, ZrO₂, TiO₂, TiO, Ti₂O₃, Ti₂O₅, Al₂O₃, Ta₂O₅, Ta₃O₅, Nb₂O₅, HfO₂, Si₃N₄, CeO₂, MgO, Y₂O₃, SnO₂, MgF₂, and WO₃. These inorganic substances may be used alone or in combination (e.g., as a mixture) of two or more of them. In the case of the multi-layer antireflection layer, it is preferred to use SiO₂ and/or SiO in the outermost layer. When the article according to the present disclosure is an optical glass component for a touch panel, a thin film using a transparent electrode, e.g., indium tin oxide (ITO), indium zinc oxide, or the like, may be provided on a part of the surface of the substrate (glass). Further, the substrate may also include an insulating layer, an adhesive layer, a protective layer, a decorative frame layer (I-CON), an atomization film layer, a hard coating film layer, a polarizing film, a phase difference film, a liquid crystal display module, or the like according to its specific specifications.

### [Method for Manufacturing Article]

A method for manufacturing an article according to the present disclosure is, for example, a method for manufacturing an article including a surface-treated layer formed on a substrate by performing a surface treatment on the substrate by using a surface treatment agent according to the present disclosure. Examples of surface treatments include a dry coating method and a wet coating method.

Examples of dry coating methods include techniques such as vacuum vapor deposition, CVD, and sputtering. As the dry coating method, a vacuum vapor-deposition method is preferred in order to suppress the decomposition of the compound and in view of the simplicity of the apparatus. During the vacuum deposition, a pellet-like substance obtained by impregnating a porous material made of a metal such as iron or steel with a compound according to the present disclosure may be used. A pellet-like substance, which is obtained by impregnating a porous material made of a metal such as iron or steel with a composition comprising a compound according to the present disclosure and a liquid medium, and drying the liquid medium, may be used. When a pellet-like substance is used, the mobility of molecules of the compound that is in a solid state at 25° C. is low, so that the deterioration of the storage stability due to the hydrolysis and the condensation reaction is suppressed.

Examples of wet coating methods include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

In order to improve the abrasion resistance of the surface-treated layer, when necessary, an operation for accelerating the reaction between the compound according to the present disclosure and the substrate may be performed. Examples of such operations include heating, humidification, and irradiation with light.

For example, it is possible to accelerate, by heating a substrate on which a surface-treated layer is formed in an atmosphere comprising moisture, the reaction such as a hydrolysis reaction of a hydrolyzable group, a reaction between a hydroxyl group or the like and a silanol group on the surface of the substrate, and formation of a siloxane bond by a condensation reaction of a silanol group.

After the surface treatment, compounds which are contained in the surface-treated layer and are not chemically bonded to other compounds nor the substrate may be removed as required. Examples of removal methods include a method in which a solvent is poured over the surface-treated layer, and a method in which the surface-treated layer or the like is wiped with a cloth impregnated with a solvent.

### EXAMPLES

The present invention will be described hereinafter in a more detailed manner by using examples, but the present invention is not limited to these examples. Note that Examples 1 to 9 are examples according to the present disclosure, and Examples 10 to 16 are comparative examples.

### Synthesis Example 1: Synthesis of Compound (1a)

Tetrahydrofuran (THF) (10 g) and copper(II) chloride (0.05 g) were added to 18-bromo-1-octadecene (1.0 g), and the mixture was stirred at 25° C. Then, a diethyl ether solution of 2,2-dimethylpropylmagnesium chloride (1.0 mol/L) (10 mL) was added to the resultant solution, and the mixture was stirred at 60° C. for 24 hours. Extraction was performed by adding hydrochloric acid and hexane, and volatiles were removed by distillation under a reduced pressure. Then, 0.6 g of compound (1a) was obtained by performing flash column chromatography using silica gel (developing solvent: hexane/di chloromethane).

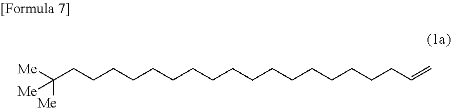 [SMILES: C=CCCCCCCCCCCCCCCCCCC(C)(C)C]

### NMR of Compound (1a)

¹H-NMR (400 MHz, CDCl₃) δ 5.80 (ddt, J=17.2, 10.3, 7.0 Hz, 1H), 5.24-4.79 (m, 2H), 2.03 (tdt, J=7.9, 6.9, 1.1 Hz, 2H), 1.53-1.10 (m, 32H), 0.85 (s, 9H).

### Synthesis Example 2: Synthesis of Compound (I)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (1a) (0.6 g) dissolved in dichloromethane (10 g), and the mixture was stirred at 50° C. for 2 hours. 0.9 g of compound (I) was obtained by removing the solvent by distillation under a reduced pressure.

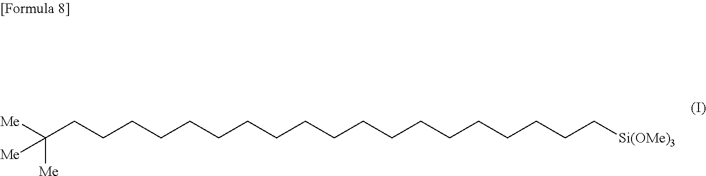

### NMR of Compound (I)

¹H-NMR (400 MHz, CDCl₃) δ 3.58 (s, 9H), 1.61-1.04 (m, 36H), 0.85 (s, 9H), 0.65 (t, J=9.2 Hz, 2H).

### Synthesis Example 3: Synthesis of Compound (2a)

THF (10 g) and copper(II) chloride (0.05 g) were added to 18-bromo-1-octadecene (1.0 g), and the mixture was stirred at 25° C. Then, a diethyl ether solution of trimethylsilyl methylmagnesium chloride (1.0 mol/L) (10 mL) was added to the resultant solution, and the mixture was stirred at 60° C. for 24 hours. Extraction was performed by adding hydrochloric acid and hexane, and volatiles were removed by distillation under a reduced pressure. Then, 0.5 g of compound (2a) was obtained by performing flash column chromatography using silica gel (developing solvent: hexane/dichloromethane).

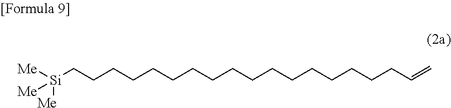 [SMILES: C=CCCCCCCCCCCCCCCCCC[Si](C)(C)C]

### NMR of Compound (2a)

¹H-NMR (400 MHz, CDCl₃) δ 5.82 (ddt, J=17.0, 10.2, 6.7 Hz, 1H), 5.19-4.75 (m, 2H), 2.04 (dt, J=8.0, 6.7 Hz, 2H), 1.53-1.10 (m, 30H), 0.60-0.27 (m, 2H), −0.03 (s, 9H)

### Synthesis Example 4: Synthesis of Compound (II)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (2a) (0.5 g) dissolved in dichloromethane (10 g), and the mixture was stirred at 50° C. for 2 hours. 0.7 g of compound (II) was obtained by removing the solvent by distillation under a reduced pressure.

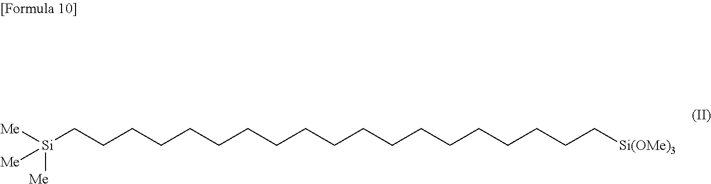

### NMR of Compound (II)

¹H-NMR (400 MHz, CDCl₃) δ 3.57 (s, 9H), 1.53-1.05 (m, 34H), 0.74-0.59 (m, 2H), 0.47 (t, J=7.3 Hz, 2H), −0.03 (s, 9H)

### Synthesis Example 5: Synthesis of Compound (3a)

THF (10 g), a toluene solution of platinum/1,3-divinyl-1,1,3,3-tetramethyldisiloxane complex (platinum content: 3 mass %, 5 mg), and chlorodimethylsilane (10 g) were added to 18-bromo-1-octadecene (10 g), and the mixture was stirred at 25° C. for 24 hours. 12 g of compound (3a) was obtained by removing volatiles by distillation under a reduced pressure.

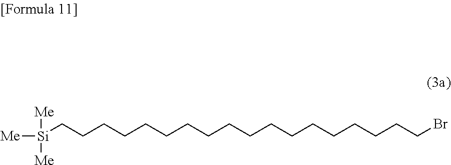 [SMILES: C[Si](C)(C)CCCCCCCCCCCCCCCCCCBr]

### NMR of Compound (3a)

¹H-NMR (400 MHz, CDCl₃) δ 3.40 (t, J=6.9 Hz, 2H), 1.92-1.74 (m, 2H), 1.46-1.06 (m, 30H), 0.88-0.71 (m, 2H), 0.40 (s, 6H).

### Synthesis Example 6: Synthesis of Compound (3b)

THF (10 g) and a THF solution of methylmagnesium chloride (1.0 mol/L) (40 mL) were added to the compound (3a) (12 g), and the mixture was stirred at 25° C. for 1 hour. Extraction was performed by adding hydrochloric acid and hexane, and volatiles were removed by distillation under a reduced pressure. Then, 8 g of compound (3b) was obtained by performing flash column chromatography using silica gel (developing solvent: hexane/dichloromethane).

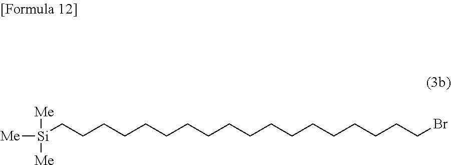 [SMILES: C[Si](C)(C)CCCCCCCCCCCCCCCCCCBr]

### NMR of Compound (3b)

¹H-NMR (400 MHz, CDCl₃) δ 3.41 (t, J=6.9 Hz, 2H), 1.85 (p, J=7.0 Hz, 2H), 1.46-1.06 (m, 30H), 0.47 (t, J=7.6 Hz, 2H), −0.03 (s, 9H).

### Synthesis Example 7: Synthesis of Compound (3c)

THF (10 g), a THF solution of allylmagnesium chloride (1.0 mol/L) (5 mL), and copper(II) chloride (0.05 g) were added to the compound (3b) (1 g), and the mixture was stirred at 60° C. for 24 hours. Extraction was performed by adding hydrochloric acid and hexane, and volatiles were removed by distillation under a reduced pressure. Then, 0.6 g of compound (3c) was obtained by performing flash column chromatography using silica gel (developing solvent: hexane/di chloromethane).

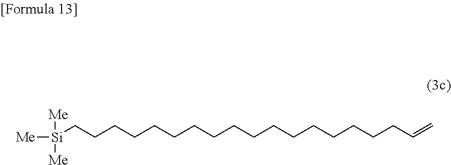 [SMILES: C=CCCCCCCCCCCCCCCCCC[Si](C)(C)C]

### NMR of Compound (3c)

¹H-NMR (400 MHz, CDCl₃) δ 5.81 (ddt, J=16.9, 10.2, 6.7 Hz, 1H), 5.10-4.77 (m, 2H), 2.11-1.82 (m, 2H), 1.46-1.06 (m, 34H), 0.48 (d, J=8.0 Hz, 2H), −0.03 (s, 9H).

### Synthesis Example 8: Synthesis of Compound (III)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (3c) (0.6 g) dissolved in dichloromethane (10 g), and the mixture was stirred at 50° C. for 2 hours. 0.8 g of compound (III) was obtained by removing the solvent by distillation under a reduced pressure.

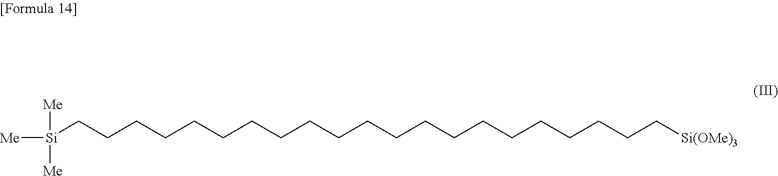

### NMR of Compound (III)

¹H-NMR (400 MHz, CDCl₃) δ 3.57 (s, 9H), 1.46-1.06 (m, 38H), 0.75-0.57 (m, 2H), 0.47 (t, J=7.7 Hz, 2H), −0.03 (s, 9H).

### Synthesis Example 9: Synthesis of Compound (4a)

THF (20 g) and magnesium (1.1 g) were added to 11-bromo-1-undecene (10 g), and the mixture was stirred at 60° C. for 2 hours. 30 g of compound (4a) was obtained by filtering the reaction solution. It was confirmed that the concentration of the product was 0.8 mol/L by titration using 1,10-phenanthroline.

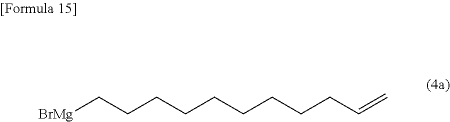

### Synthesis Example 10: Synthesis of Compound (4b)

THF (10 g), the compound (4a) (0.8 mol/L) (10 mL), and copper(II) chloride (0.05 g) were added to the compound (3b) (1 g), and the mixture was stirred at 60° C. for 24 hours. Extraction was performed by adding hydrochloric acid and hexane, and volatiles were removed by distillation under a reduced pressure. Then, 0.4 g of compound (4b) was obtained by performing flash column chromatography using 10% silver nitrate silica gel (developing solvent: hexane/dichloromethane).

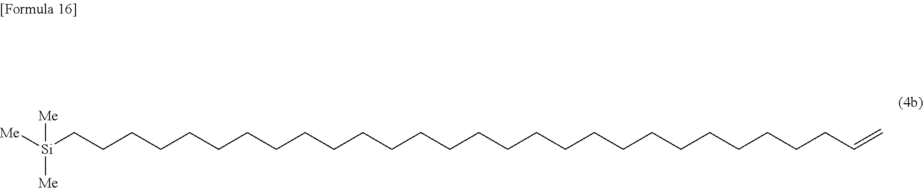 [SMILES: C=CCCCCCCCCCCCCCCCCCCCCCCCCCCC[Si](C)(C)C]

### NMR of Compound (4b)

¹H-NMR (400 MHz, CDCl₃) δ 5.82 (ddt, J=16.9, 10.2, 6.7 Hz, 1H), 5.09-4.78 (m, 2H), 2.04 (q, J=7.1 Hz, 2H), 1.46-1.06 (m, 50H), 0.47 (t, J=7.7 Hz, 2H), −0.03 (s, 9H).

### Synthesis Example 11: Synthesis of Compound (IV)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (4b) (0.4 g) dissolved in dichloromethane (10 g), and the mixture was stirred at 50° C. for 2 hours. 0.5 g of compound (IV) was obtained by removing the solvent by distillation under a reduced pressure.

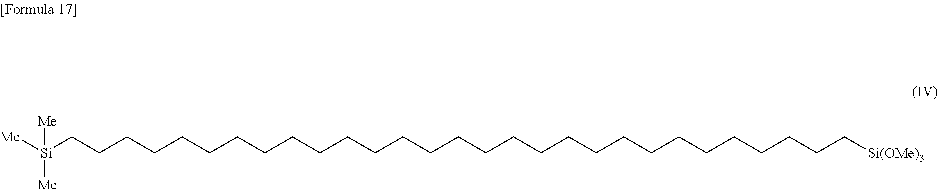

### NMR of Compound (IV)

¹H-NMR (400 MHz, CDCl₃) δ 3.57 (s, 9H), 1.46-1.06 (m, 54H), 0.72-0.59 (m, 2H), 0.48 (d, J=8.0 Hz, 2H), −0.03 (s, 9H).

### Synthesis Example 12: Synthesis of Compound (5a)

THF (40 g) and magnesium (0.9 g) were added to 18-bromo-1-octadecene (10 g), and the mixture was stirred at 60° C. for 2 hours. 50 g of compound (5a) was obtained by filtering the reaction solution. It was confirmed that the concentration of the product was 0.4 mol/L by titration using 1,10-phenanthroline.

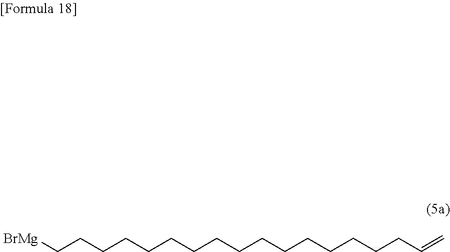

### Synthesis Example 13: Synthesis of Compound (5b)

THF (10 g), the compound (5a) (0.4 mol/L) (20 mL), and copper(II) chloride (0.05 g) were added to the compound (3b) (1 g), and the mixture was stirred at 60° C. for 24 hours. Extraction was performed by adding hydrochloric acid and hexane, and volatiles were removed by distillation under a reduced pressure. Then, 0.4 g of compound (5b) was obtained by performing flash column chromatography using 10% silver nitrate silica gel (developing solvent: hexane/dichloromethane).

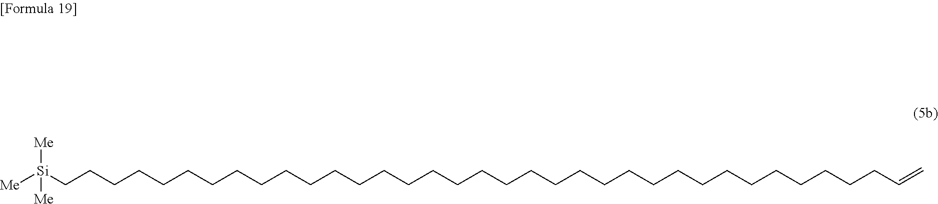 [SMILES: C=CCCCCCCCCCCCCCCCCCCCCCCCCCCCCCCCCCC[Si](C)(C)C]

### NMR of Compound (5b)

¹H-NMR (400 MHz, CDCl₃) δ 5.80 (ddt, J=17.2, 10.2, 7.0 Hz, 1H), 5.21-4.83 (m, 2H), 2.03 (tdt, J=7.8, 6.8, 1.1 Hz, 2H), 1.49-1.05 (m, 64H), 0.58 (t, J=8.5 Hz, 2H), −0.03 (s, 9H).

### Synthesis Example 14: Synthesis of Compound (V)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (5b) (0.4 g) dissolved in dichloromethane (10 g), and the mixture was stirred at 50° C. for 2 hours. 0.4 g of compound (V) was obtained by removing the solvent by distillation under a reduced pressure.

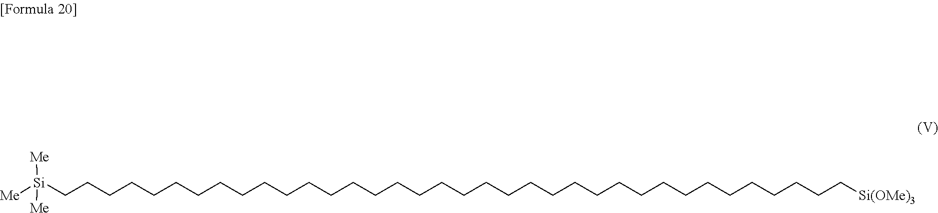

### NMR of Compound (V)

¹H-NMR (400 MHz, CDCl₃) δ 3.59 (s, 9H), 1.46-1.06 (m, 68H), 0.72-0.59 (m, 2H), 0.53-0.38 (m, 2H), −0.03 (s, 9H).

### Synthesis Example 15: Synthesis of Compound (6a)

THF (10 g), a toluene solution of platinum/1,3-divinyl-1,1,3,3-tetramethyldisiloxane complex (platinum content: 3 mass %, 5 mg), and dichloromethylsilane (10 g) were added to 18-bromo-1-octadecene (10 g), and the mixture was stirred at 25° C. for 24 hours. 13 g of compound (6a) was obtained by removing volatiles by distillation under a reduced pressure.

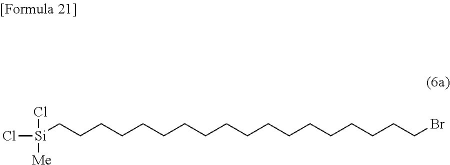 [SMILES: C[Si](Cl)(Cl)CCCCCCCCCCCCCCCCCCBr]

### NMR of Compound (6a)

¹H-NMR (400 MHz, CDCl₃) δ 3.41 (t, J=6.9 Hz, 2H), 1.85 (p, J=7.0 Hz, 2H), 1.57-1.02 (m, 32H), 0.77 (s, 3H).

### Synthesis Example 16: Synthesis of Compound (6b)

THF (10 g) and a diethyl ether solution of trimethylsilyl methylmagnesium chloride (1.0 mol/L) (10 mL) were added to the compound (6a) (1 g), and the mixture was stirred at 10° C. for 2 hours. Extraction was performed by adding hydrochloric acid and hexane, and volatiles were removed by distillation under a reduced pressure. Then, 0.8 g of compound (6b) was obtained by performing flash column chromatography using silica gel (developing solvent: hexane/di chloromethane).

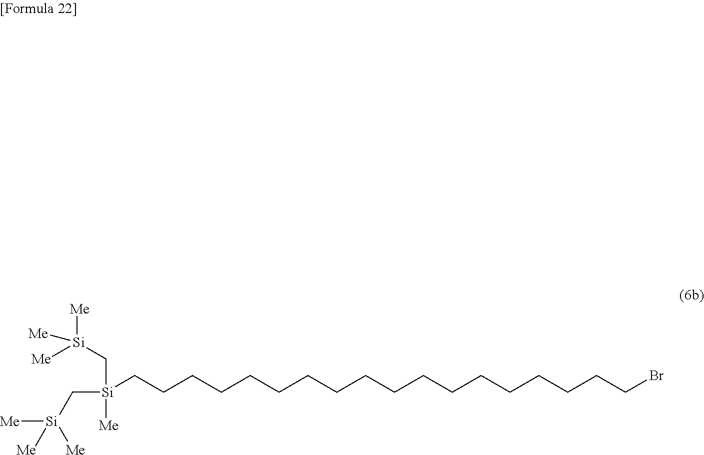 [SMILES: C[Si](C)(C)C[Si](C)(CCCCCCCCCCCCCCCCCCBr)C[Si](C)(C)C]

### NMR of Compound (6b)

¹H-NMR (400 MHz, CDCl₃) δ 3.41 (t, J=6.9 Hz, 2H), 1.85 (dt, J=14.4, 7.0 Hz, 2H), 1.57-1.02 (m, 30H), 0.61-0.40 (m, 2H), 0.06 (s, 21H), −0.20-−0.38 (m, 4H).

### Synthesis Example 17: Synthesis of Compound (6c)

THF (10 g), a THF solution of allylmagnesium chloride (1.0 mol/L) (5 mL), and copper(II) chloride (0.05 g) were added to the compound (6b) (0.8 g), and the mixture was stirred at 60° C. for 24 hours. Extraction was performed by adding hydrochloric acid and hexane, and volatiles were removed by distillation under a reduced pressure. Then, 0.6 g of compound (6c) was obtained by performing flash column chromatography using silica gel (developing solvent: hexane/di chloromethane).

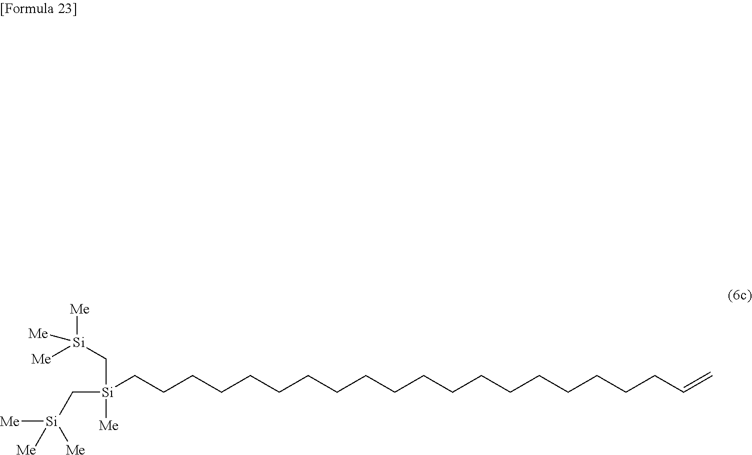 [SMILES: C=CCCCCCCCCCCCCCCCCCCC[Si](C)(C[Si](C)(C)C)C[Si](C)(C)C]

### NMR of Compound (6c)

¹H-NMR (400 MHz, CDCl₃) δ 5.80 (ddt, J=17.8, 10.6, 7.4 Hz, 1H), 5.19-4.77 (m, 2H), 1.98 (dt, J=7.5, 1.3 Hz, 2H), 1.57-1.02 (m, 34H), 0.49 (d, J=9.8 Hz, 2H), 0.06 (s, 21H), −0.28 (d, J=1.7 Hz, 4H).

### Synthesis Example 18: Synthesis of Compound (VI)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (6c) (0.6 g) dissolved in dichloromethane (10 g), and the mixture was stirred at 50° C. for 2 hours. 0.7 g of compound (VI) was obtained by removing the solvent by distillation under a reduced pressure.

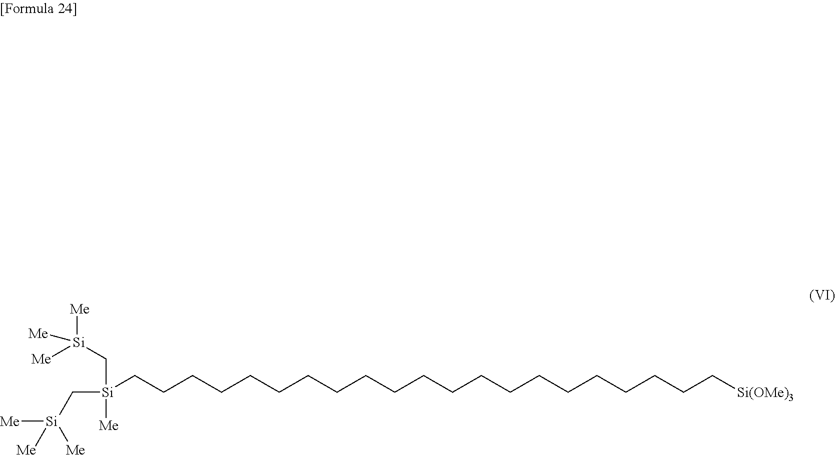

### NMR of Compound (VI)

¹H-NMR (400 MHz, CDCl₃) δ 3.56 (s, 9H), 1.38-0.96 (m, 38H), 0.65-0.35 (m, 4H), 0.15-−0.07 (m, 21H), −0.28 (d, J=1.7 Hz, 4H).

### Synthesis Example 19: Synthesis of Compound (XIV)

A toluene solution of platinum/1,3-divinyl-1,1,3,3-tetramethyldisiloxane complex (platinum content: 3 mass %, 5 mg), triacetoxymethylsilane (3 mg), and trichlorosilane (0.6 g) were added to the compound (5b) (0.4 g) dissolved in dichloromethane (10 g), and the mixture was stirred at 30° C. for 2 hours. After removing the solvent by distillation under a reduced pressure, 2-(trimethylsilyl) ethanol (3.0 g), triethylamine (3.0 g), and dichloromethane (10 g) were added to the resultant solution, and the mixture was stirred at 30° C. for 2 hours. After removing the solvent by distillation under a reduced pressure, 0.7 g of compound (XIV) was obtained by performing flash column chromatography using silica gel (developing solvent: hexane/di chloromethane).

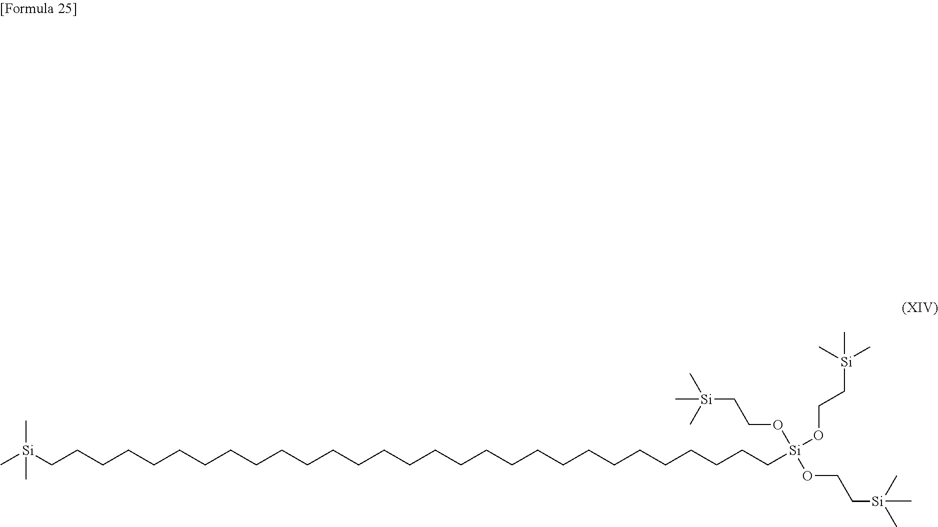

### NMR of Compound (XIV)

¹H-NMR (400 MHz, CDCl3) δ 4.08-3.58 (m, 6H), 1.47-0.78 (m, 60H), 0.70-0.54 (m, 2H), 0.46 (t, J=7.3 Hz, 2H), −0.06 (s, 36H).

### Synthesis Example 20: Synthesis of Compound (XV)

A toluene solution of platinum/1,3-divinyl-1,1,3,3-tetramethyldisiloxane complex (platinum content: 3 mass %, 5 mg), triacetoxymethylsilane (3 mg), and trichlorosilane (0.6 g) were added to the compound (5b) (0.4 g) dissolved in dichloromethane (10 g), and the mixture was stirred at 30° C. for 2 hours. After removing the solvent by distillation under a reduced pressure, ethanol (3.0 g), triethylamine (3.0 g), and dichloromethane (10 g) were added to the resultant solution, and the mixture was stirred at 30° C. for 2 hours. After removing the solvent by distillation under a reduced pressure, 0.6 g of compound (XV) was obtained by performing flash column chromatography using silica gel (developing solvent: hexane/di chloromethane).

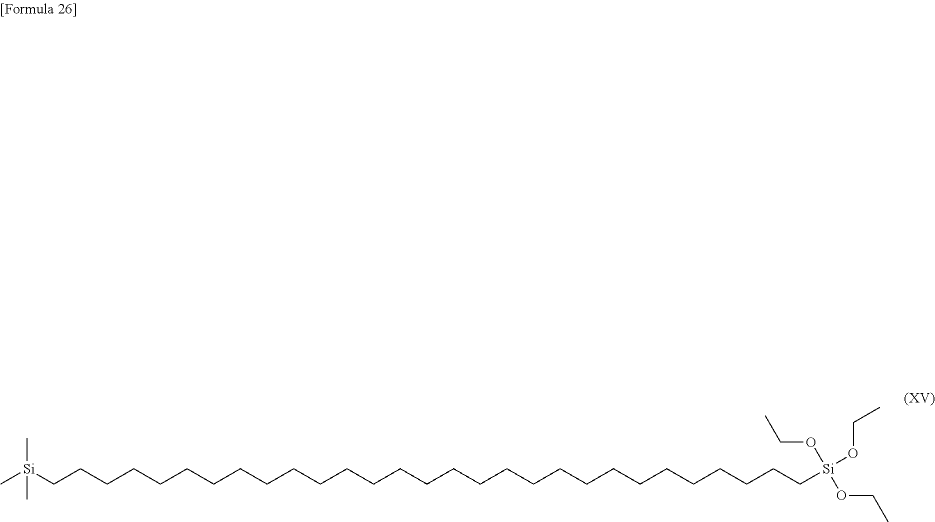 [SMILES: CCO[Si](CCCCCCCCCCCCCCCCCCCCCCCCCCCCC[Si](C)(C)C)(OCC)OCC]

### NMR of Compound (XV)

¹H-NMR (400 MHz, CDCl₃) δ 3.82 (q, J=7.0 Hz, 6H), 1.24 (d, J=10.0 Hz, 63H), 0.70-0.56 (m, 2H), 0.47 (t, J=7.7 Hz, 2H) −0.06 (s, 9H).

### Synthesis Example 21: Synthesis of Compound (XVI)

A toluene solution of platinum/1,3-divinyl-1,1,3,3-tetramethyldisiloxane complex (platinum content: 3 mass %, 5 mg), triacetoxymethylsilane (3 mg), and trichlorosilane (0.6 g) were added to the compound (5b) (0.4 g) dissolved in dichloromethane (10 g), and the mixture was stirred at 30° C. for 2 hours. After removing the solvent by distillation under a reduced pressure, 3,3-dimethyl-1-butanol (3.0 g), triethylamine (3.0 g), and dichloromethane (10 g) were added to the resultant solution, and the mixture was stirred at 30° C. for 2 hours. After removing the solvent by distillation under a reduced pressure, 0.7 g of compound (XVI) was obtained by performing flash column chromatography using silica gel (developing solvent: hexane/di chloromethane).

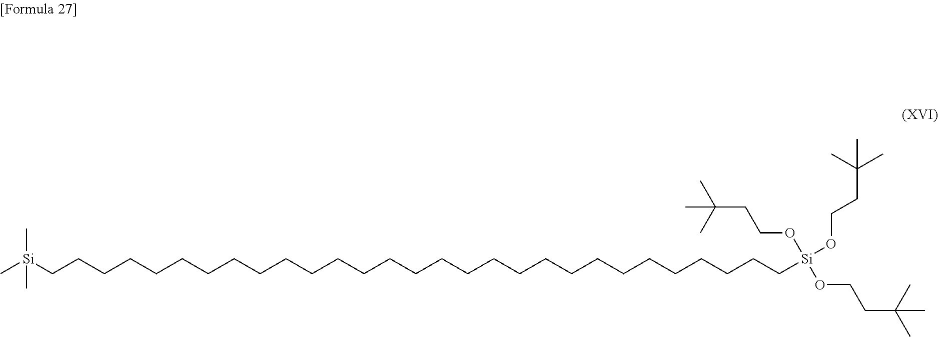 [SMILES: CC(C)(C)CCO[Si](CCCCCCCCCCCCCCCCCCCCCCCCCCCCC[Si](C)(C)C)(OCCC(C)(C)C)OCCC(C)(C)C]

NMR of compound (XVI)

¹H-NMR (400 MHz, CDCl₃) δ 3.68 (t, J=8.1 Hz, 6H), 1.60 (t, J=8.2 Hz, 6H), 1.50-0.42 (m, 85H), −0.13 (s, 9H).

### Synthesis Example 22: Synthesis of Compound (VII)

The following compound (VII) was obtained by a method disclosed in International Patent Publication No. WO2023/017830. The average value of the numbers n of repetitive units was 17.

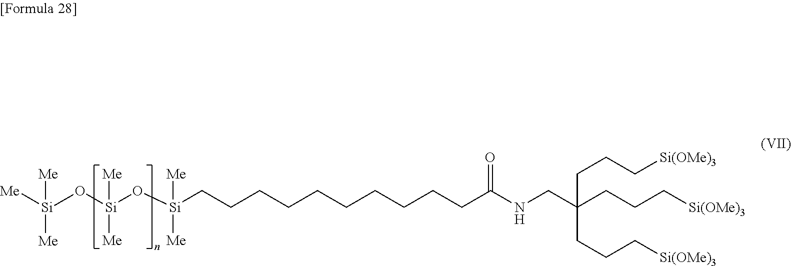

### [Compound (VIII), Compound (IX)]

The following commercially available compounds were used as compound (VIII) and compound (IX), respectively. The average value of the numbers n of repetitive units of the compound (VIII) was 62.

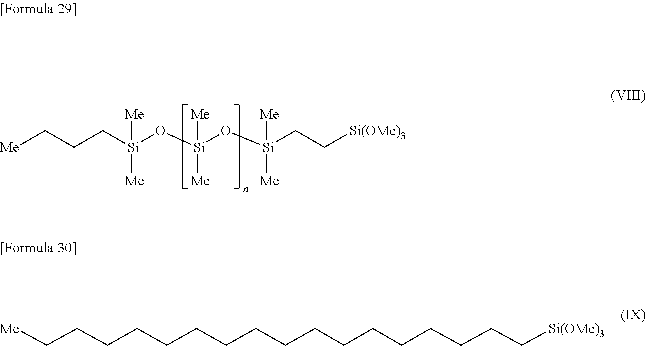

### Synthesis Example 23: Synthesis of Compound (10a)

THF (10 g) and a diethyl ether solution of methyllithium (1.0 mol/L) (4 mL) were added to tris(trimethylsilyl) methane (1.0 g), and the mixture was stirred at 50° C. for 1 hour. Then, 18-bromo-1-octadecene (1.0 g) was added to the resultant solution, and the mixture was stirred at 50° C. for 24 hours. Extraction was performed by adding hydrochloric acid and hexane, and volatiles were removed by distillation under a reduced pressure. Then, 0.3 g of compound (10a) was obtained by performing flash column chromatography using silica gel (developing solvent: hexane/dichloromethane).

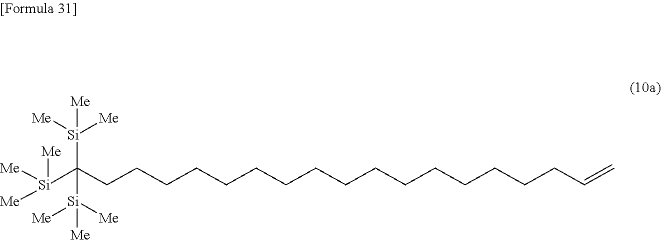 [SMILES: C=CCCCCCCCCCCCCCCCCC([Si](C)(C)C)([Si](C)(C)C)[Si](C)(C)C]

### NMR of Compound (10a)

¹H-NMR (400 MHz, CDCl₃) δ 5.80 (ddt, J=17.2, 10.3, 7.0 Hz, 1H), 5.19-4.86 (m, 2H), 2.03 (tdt, J=7.8, 6.8, 1.1 Hz, 2H), 1.56-0.70 (m, 30H), −0.01 (s, 27H).

### Synthesis Example 24: Synthesis of Compound (X)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (10a) (0.3 g) dissolved in dichloromethane (10 g), and the mixture was stirred at 50° C. for 2 hours. 0.4 g of compound (X) was obtained by removing the solvent by distillation under a reduced pressure.

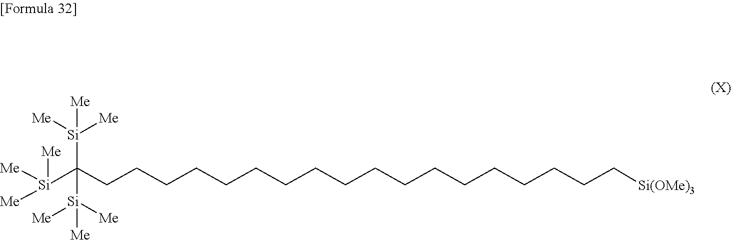

### NMR of Compound (X)

¹H-NMR (400 MHz, CDCl3) δ 3.58 (s, 9H), 1.53-0.51 (m, 36H), −0.01 (s, 27H).

### Synthesis Example 25: Compound (11a)

THF (10 g) and copper(II) chloride (0.05 g) were added to 7-bromo-1-heptene (1.0 g), and the mixture was stirred at 25° C. Then, a diethyl ether solution of 2,2-dimethylpropylmagnesium chloride (1.0 mol/L) (20 mL) was added to the resultant solution, and the mixture was stirred at 60° C. for 24 hours. Extraction was performed by adding hydrochloric acid and hexane, and volatiles were removed by distillation under a reduced pressure. Then, 0.5 g of compound (11a) was obtained by performing flash column chromatography using silica gel (developing solvent: hexane/dichloromethane).

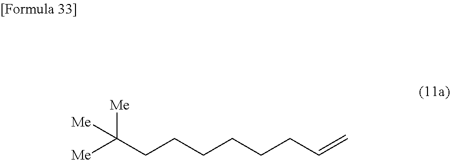

### NMR of Compound (11a)

¹H-NMR (400 MHz, CDCl₃) δ 5.80 (ddt, J=17.2, 10.3, 7.0 Hz, 1H), 5.21-4.70 (m, 2H), 2.03 (tdt, J=7.7, 6.7, 1.0 Hz, 2H), 1.42-0.58 (m, 19H).

### Synthesis Example 26: Synthesis of Compound (XI)

A toluene solution of platinum/1,3-divinyl-1,1,3,3-tetramethyldisiloxane complex (platinum content: 3 mass %, 5 mg), aniline (3 mg), and trimethoxysilane (0.60 g) were added to the compound (11a) (0.5 g) dissolved in dichloromethane (10 g), and the mixture was stirred at 50° C. for 2 hours. 0.7 g of compound (XI) was obtained by removing the solvent by distillation under a reduced pressure.

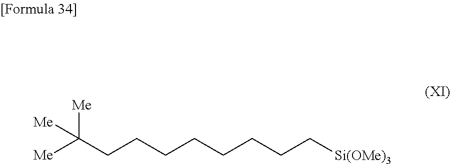

### NMR of Compound (XI)

¹H-NMR (400 MHz, CDCl₃) δ 3.58 (s, 9H), 1.53-1.11 (m, 14H), 0.85 (s, 9H), 0.65 (t, J=9.2 Hz, 2H).

### Synthesis Example 27: Synthesis of Compound (12a)

A THF solution of compound (12a) (0.8 mol/L) was obtained by a method disclosed in International Patent Publication No. WO2021/054413.

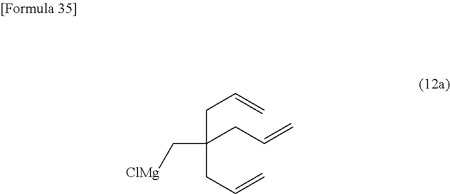

### Synthesis Example 28: Synthesis of Compound (12b)

1,18-octadecanediol (25 g) was dissolved in toluene (1,000 mL). 48% hydrobromic acid (22 g) was added to the resultant solution, and the mixture was stirred for 24 hours while being refluxed. After the completion of the reaction, the solution was separated, and the aqueous layer was extracted with dichloromethane. The organic layer was collected and washed with saturated sodium bicarbonate solution. The resultant substance was dried over anhydrous magnesium sulfate and filtered. 22 g of compound (12b) was obtained by removing the filtrate by distillation under a reduced pressure and purifying the resultant substance by silica gel column chromatography.

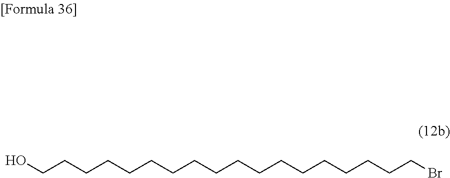 [SMILES: OCCCCCCCCCCCCCCCCCCBr]

### NMR of Compound (12b)

¹H-NMR (400 MHz, CDCl₃) δ 3.57 (td, J=5.8, 5.8 Hz, 2H), 3.42 (t, J=4.7 Hz, 2H), 1.81-1.20 (m, 32H).

### Synthesis Example 29: Synthesis of Compound (12c)

The compound (12b) (21 g) was dissolved in THF (500 mL); copper(II) chloride (0.4 g) and a tetrahydrofuran solution (0.8 mol/L) of the compound (12a) (200 mL) were added; and the mixture was stirred at 50° C. for 24 hours. After the completion of the reaction, hydrochloric acid was added, and extraction was performed with dichloromethane. The solvent and volatiles were removed by distillation under a reduced pressure, and 18 g of compound (12c) was obtained by performing flash column chromatography using silica gel.

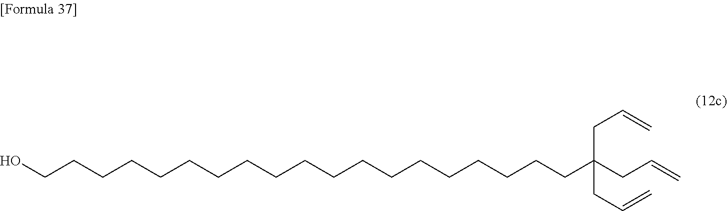 [SMILES: C=CCC(CC=C)(CC=C)CCCCCCCCCCCCCCCCCCCO]

### NMR of Compound (12c)

¹H-NMR (400 MHz, CDCl₃) δ 5.81-5.57 (m, 3H), 5.28-4.88 (m, 6H), 3.57 (td, J=5.8, 5.8 Hz, 2H), 1.90-1.78 (m, 6H), 1.64-1.48 (m, 2H), 1.38-1.15 (m, 32H).

### Synthesis Example 30: Synthesis of Compound (12d)

The compound (12c) (17 g) was dissolved in THF (500 mL), and triphenylphosphine (20 g) was added to the resultant solution. Carbon tetrabromide (20 g) was added little by little while stirring the solution in an ice bath, and the stirring was continued for 24 hours. Silica gel was added to the reaction solution, and the solvent was removed by distillation. 14 g of compound (12d) was obtained by extracting it with hexane.

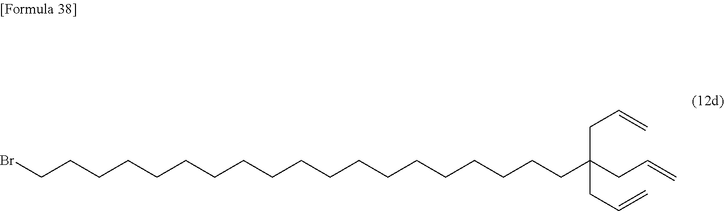 [SMILES: C=CCC(CC=C)(CC=C)CCCCCCCCCCCCCCCCCCCBr]

### NMR of Compound (12d)

¹H-NMR (400 MHz, CDCl₃) δ 5.80-5.59 (m, 3H), 5.23-4.90 (m, 6H), 3.42 (t, J=4.7 Hz, 2H), 1.85 (d, J=7.3 Hz, 6H), 1.82-1.10 (m, 36H).

### Synthesis Example 31: Synthesis of Compound (12e)

The compound (12d) (5.0 g) was dissolved in THF (100 mL); copper(II) chloride (0.005 g) and a diethyl ether solution of trimethylsilyl methylmagnesium chloride (1.0 mol/L) (16 mL) were added; and the mixture was stirred at 60° C. for 24 hours. After the completion of the reaction, hydrochloric acid was added, and extraction was performed with hexane. The solvent and volatiles were removed by distillation under a reduced pressure, and 2.5 g of compound (12e) was obtained by performing flash column chromatography using silica gel.

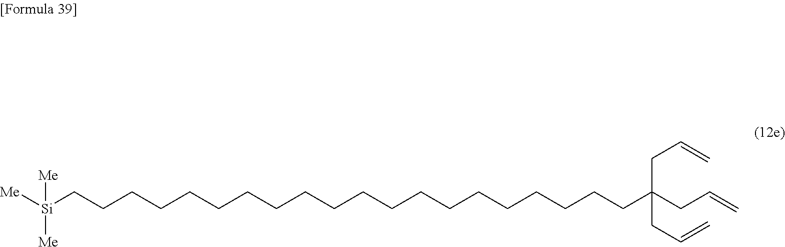 [SMILES: C=CCC(CC=C)(CC=C)CCCCCCCCCCCCCCCCCCCC[Si](C)(C)C]

### NMR of Compound (12e)

¹H-NMR (400 MHz, CDCl₃) δ 5.70 (ddt, J=16.8, 11.3, 7.4 Hz, 3H), 5.23-4.85 (m, 6H), 1.84 (dt, J=7.3, 1.0 Hz, 6H), 1.45-1.10 (m, 38H), 0.58 (t, J=8.5 Hz, 2H), −0.05 (s, 9H).

### Synthesis Example 32: Synthesis of Compound (XII)

A toluene solution of platinum/1,3-divinyl-1,1,3,3-tetramethyldisiloxane complex (platinum content: 3 mass %, 5 mg), aniline (3 mg), and trimethoxysilane (1.0 g) were added to the compound (12e) (0.5 g) dissolved in dichloromethane (10 g), and the mixture was stirred at 50° C. for 2 hours. 0.7 g of compound (XII) was obtained by removing the solvent by distillation under a reduced pressure.

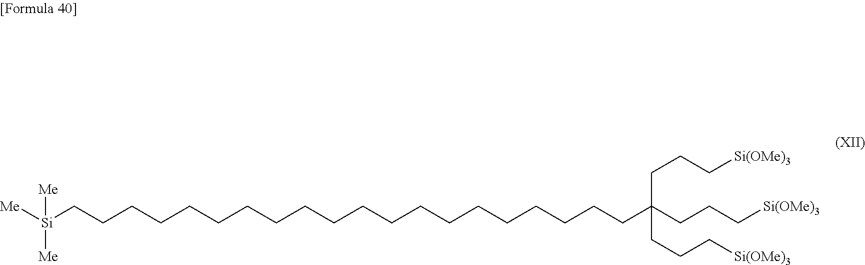

### NMR of Compound (XII)

¹H-NMR (400 MHz, CDCl₃) δ 3.58 (s, 27H), 1.51-1.08 (m, 50H), 0.77-0.46 (m, 8H), −0.05 (s, 9H).

### Synthesis Example 33: Synthesis of Compound (XIII)

Compound (XIII) was obtained with reference to Synthesis Examples 1 to 11 disclosed in International Patent Publication No. WO2023/181867.

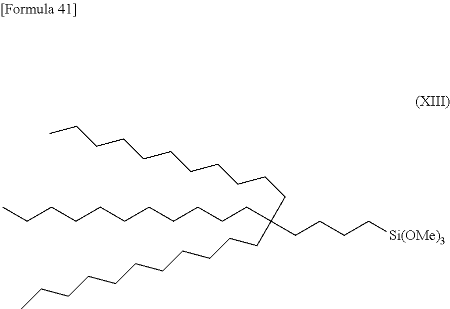

### [Manufacturing of Article]

For each of the above-described compounds, a surface treatment was performed on a substrate by using the compound. A dry coating method was used as the surface treatment method. A chemically reinforced glass was used as the substrate.

### <Dry Coating Method>

Dry coating was carried out by using a vacuum vapor-deposition apparatus (VTR-350M, manufactured by ULVAC, Inc.). 30 g of silicon oxide was disposed as a vapor-deposition source on a copper hearth in a vacuum vapor-deposition apparatus (“VTR-350M” manufactured by ULVAC KIKO, Inc.). A glass substrate was disposed in the vacuum vapor-deposition apparatus, and the vacuum vapor-deposition apparatus was evacuated of air so that the pressure inside the apparatus became 5×10⁻³ Pa or lower. A substrate with a silicon oxide layer having a thickness of about 20 nm was prepared by heating the above-described hearth to about 2,000° C., and vapor-depositing silicon oxide on the surface of the substrate in a vacuum.

For each of the compounds, 10% heptane solution (0.5 g) of the compound was charged into a boat made of molybdenum in the vacuum vapor-deposition apparatus, and the vacuum vapor-deposition apparatus was evacuated of air so that the pressure inside the apparatus became 1×10⁻³ Pa or lower. The boat was heated at a temperature rising rate of 10° C./min or lower. Then, at the moment when the vapor-deposition rate measured by a quartz oscillation-type film thickness gauge exceeded 1 nm/sec, the shutter was opened and film formation (i.e., the deposition of the compound) on the surface of the substrate was thereby started. When the film thickness became about 50 nm, the shutter was closed and the film formation on the surface of the substrate with the silicon oxide layer was thereby finished. The substrate on which the compound was deposited was heat-treated at 200° C. for 30 minutes, and an article having a surface-treated layer on the surface of the substrate was thereby obtained.

### [Evaluations]

The articles obtained by the dry coating method were evaluated for water repellency, abrasion resistance, and fingerprint removal property. The evaluation methods were as follows.

### <Initial Contact Angle (Water Repellency)>

About 2 μL of distilled water was added dropwise on the surface-treated layer of the article, and the initial water contact angle was measured by using a contact angle measuring apparatus (product name: DM-500 manufactured by Kyowa Interface Science Co., Ltd). An average value of values measured at five points on the surface-treated layer was defined as the water contact angle. The water repellency of the surface-treated layer was evaluated based on criteria shown below. Note that a 2θ method was used for the calculation of the water contact angle.
A: The initial water contact angle is 105° or larger
B: The initial water contact angle is equal to or larger than 100°, and less than 105°
C: The initial water contact angle is less than 100°

### <Initial Contact Angle (Oil Repellency)>

About 2 μL of oleic acid was added dropwise on the surface-treated layer of the article, and the initial oleic-acid contact angle was measured by using a contact angle measuring apparatus (Product Name: DM-500, manufactured by Kyowa Interface Science Co., Ltd.). An average value of values measured at five points on the surface-treated layer was defined as the oleic acid contact angle. The oil repellency of the surface-treated layer was evaluated based on evaluation criteria shown below. Note that a 2θ method was used for the calculation of the oleic acid contact angle.
A: The initial oleic acid contact angle is 50° or larger
B: The initial oleic acid contact angle is equal to or larger than 40°, and less than 50°
C: The initial oleic acid contact angle is less than 40°

### <Acid Resistance>

The article was cut into 1 cm×1 cm square, and submerged in an aqueous hydrochloric acid solution (0.1 mol/L) at 20° C. for 100 hours. After that, the article was taken out from the solution, washed with ethanol, and dried. Then, the water contact angle was measured. The smaller the decrease in water repellency (water contact angle) after the submersion is, the smaller the decrease in performance due to the acid is, and hence the more excellent the acid resistance is. The evaluation criteria are as follows.
A: The decrease in water contact angle after the submersion is 3° or smaller
B: The decrease in water contact angle after the submersion is larger than 3°, and equal to or less than 5°
C: The decrease in water contact angle after the submersion is larger than 5°, and equal to or less than 10°
D: The decrease in water contact angle after the submersion is larger than 10°

### <Hot-Water Resistance>

The article was cut into 1 cm×1 cm square, and submerged in water (50cc) at 80° C. for 100 hours. After that, the water contact angle was measured. The smaller the decrease in water repellency (water contact angle) after the submersion is, the smaller the decrease in performance due to hot water is, and hence the more excellent the hot-water resistance is. The evaluation criteria are as follows.
A: The decrease in water contact angle after the submersion is 3° or smaller
B: The decrease in water contact angle after the submersion is larger than 3°, and equal to or less than 5°
C: The decrease in water contact angle after the submersion is larger than 5°, and equal to or less than 10°
D: The decrease in water contact angle after the submersion is larger than 10°

Table 1 shows evaluation results.

TABLE 1

| Example | | | 1 | 2 | 3 | 4 | 5 | 6 | 7 | 8 | 9 |
|---|---|---|---|---|---|---|---|---|---|---|---|
| Surface treatment | | Compound No. | I | II | III | IV | V | VI | XIV | XV | XVI |
| method | | Phase at 25° C. | Liquid | Liquid | Solid | Solid | Solid | Liquid | Liquid | Solid | Liquid |
| Dry | Initial contact | Water | A | A | A | A | A | A | A | A | A |
| coating | angle | Oleic acid | B | A | A | A | A | A | A | A | A |

| | | | | | | | | | |
|---|---|---|---|---|---|---|---|---|---|
| Acid resistance | B | B | B | A | A | B | A | A | A |
| Water resistance | B | B | B | A | A | B | A | A | A |

| Example | | | 10 | 11 | 12 | 13 | 14 | 15 | 16 |
|---|---|---|---|---|---|---|---|---|---|
| Surface treatment | | Compound No. | VII | VIII | IX | X | XI | XII | XIII |
| method | | Phase at 25° C. | Liquid | Liquid | Liquid | Liquid | Liquid | Liquid | Liquid |
| Dry | Initial contact | Water | A | A | C | C | B | C | A |
| coating | angle | Oleic acid | A | A | C | C | C | C | C |

| | | | | | | | |
|---|---|---|---|---|---|---|---|
| Acid resistance | D | D | B | C | D | B | D |
| Water resistance | D | D | B | C | D | B | C |

As shown in Table 1, it was clearly shown that the surface-treated layers of the articles of Examples 1 to 9 using the compounds I to VI and XIV to XVI, respectively, had excellent water and oil repellency, excellent acid resistance, and excellent hot-water resistance.

Note that among the compounds I to VI and XIV to XVI, the compounds III to V and XV were in a solid state at 25° C., and the compounds I to II, VI to XIV, and XVI were in a liquid state at 25° C.

A compound according to the present disclosure is useful as a surface treatment agent. Such a surface treatment agent can be used, for example, for substrates in display devices such as touch panel displays, optical elements, semiconductor elements, building materials, automobile components, and nanoimprinting technologies. Further, such a surface treatment agent can be used for bodies, window glasses (front glasses, side glasses, and rear glasses), mirrors, bumpers, and the like in transportation apparatuses such as trains, automobiles, ships, and airplanes. Further, such a surface treatment agent can be used for exterior walls of buildings, tents, photovoltaic modules, sound insulating plates, and outdoor articles such as concrete; and fishing nets, sweep nets, and water tanks. Further, such a surface treatment agent can be used for kitchens, bathrooms, washstands, mirrors, and toiletry components; chandeliers and ceramics such as tiles; and artificial marble and various indoor apparatuses such as air conditioners. Further, such a surface treatment agent can be used for antifouling treatments of jigs, inner walls, pipes, and the like in factories. Further, such a surface treatment agent can be used for goggles, glasses, helmets, pachinko, fibers, umbrellas, play equipment, and soccer balls. Further, such a surface treatment agent can be used as adherents for various packaging materials such as packaging materials for foods, packaging materials for cosmetics, and the interior of pots. Further, such a surface treatment agent can be used for car navigation systems, mobile phones, smart phones, digital cameras, digital video cameras, PDAs, portable audio players, car audios, game apparatuses, lenses for eyeglasses, camera lenses, lens filters, sunglasses, medical apparatuses such as gastroscopes, copy machines, PCs, displays (e.g., liquid crystal displays, organic EL displays, plasma displays, and touch panel displays), touch panels, protective films, and optical components such as antireflection films.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A compound represented by following Formula (1) 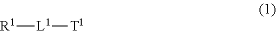
in Formula (1),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3,
L¹ is an alkylene group having 13 or more carbon atoms, and may have one or more groups selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms,
T¹ is —Si(R¹¹)ₘ₂L₃₋ₘ₂,
R¹¹ is each independently a hydrocarbon group,
L is each independently a hydrolyzable group, a group having a hydrolyzable group, or a hydroxyl group, and
m2 is an integer from 0 to 2.

2. The compound according to claim 1, wherein the number of carbon atoms of L1 is 36 or less.

3. The compound according to claim 1, wherein L1 is an alkylene group having 13 to 36 carbon atoms.

4. The compound according to claim 1, wherein R¹ is Q²(CH₃)₃—.

5. The compound according to claim 1, wherein L is represented by following Formula (G1). 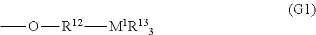
where
R¹² is an alkylene group, (Si(CH₃)₂O)ₘ₃, or a combination of an alkylene group and (Si(CH₃)₂O)ₘ₃,
M¹ is a carbon atom or a silicon atom,
R¹³ is each independently a hydrogen atom or an alkyl group which may have a branch, and each of at least two of R¹³ is an alkyl group which may have a branch, and
m3 is an integer from 1 to 12.

6. The compound according to claim 5, wherein R¹² is a linear alkylene group.

7. The compound according to claim 5, wherein M¹R¹³₃ is CH(CH₃)₂, C(CH₃)₃, or Si(CH₃)₃.

8. The compound according to claim 1, wherein the compound is in a solid state at 25° C.

9. The compound according to claim 1 wherein the compound is in a liquid state at 25° C.

10. A composition comprising a compound according to claim 1 and a liquid medium.

11. A surface treatment agent comprising a compound according to claim 1.

12. The surface treatment agent according to claim 11, further comprising a liquid medium.

13. The surface treatment agent according to claim 11, wherein the surface treatment agent is one for vacuum deposition.

14. The surface treatment agent according to claim 12, wherein the surface treatment agent is one for vacuum deposition.

15. The surface treatment agent according to claim 12, wherein the surface treatment agent is one for wet coating.

16. A pellet comprising the surface treatment agent according to claim 11.

17. A pellet comprising the surface treatment agent according to claim 12.

18. A method for manufacturing an article including a surface-treated layer formed on a substrate by performing a surface treatment on the substrate by using a surface treatment agent according to claim 11.

19. A method for manufacturing an article including a surface-treated layer formed on a substrate by performing a surface treatment on the substrate by using a surface treatment agent according to claim 12.

20. An article comprising a substrate, and a surface-treated layer disposed on the substrate, a surface of the surface-treated layer being treated with a surface treatment agent according to claim 11.

21. An article comprising a substrate, and a surface-treated layer disposed on the substrate, a surface of the surface-treated layer being treated with a surface treatment agent according to claim 12.

22. The article according to claim 20, wherein the article is an optical member.

23. The article according to claim 21, wherein the article is an optical member.

24. The article according to claim 20, wherein the article is a display or a touch panel.

25. The article according to claim 21, wherein the article is a display or a touch panel.

26. A compound represented by following Formula (13) 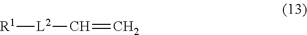
in Formula (13),
R¹ is [(CH₃)₃Q¹-R²-]ₘ₁Q²(CH₃)₃₋ₘ₁—,
Q¹ and Q² are each independently C or Si,
R² is each independently an alkylene group having 1 to 6 carbon atoms,
m1 is an integer from 0 to 3, and
L² is an alkylene group having 11 or more carbon atoms, and may have one or more bonds selected from —O—, —S—, —C(═O)NH—, —NHC(═O)—, —C(═O)O—, —OC(═O)—, —C(═O)S—, —SC(═O)—, —S(═O)₂NH—, —NHS(═O)₂—, —S(═O)₂O—, —OS(═O)₂—, and a phenylene group between carbon atoms.
